SEQUENCE LISTING (9 sequences)

SEQ ID NO: 1 - DNA, 1557 bp, Helicobacter pylori
  CDS (1)..(1554)
atgacacaatctcaaaaagtaagattcttagcccctttaagcctagcgttaagcttgagcttcaatccagtgggcgctgaagaagatgggggctttatgacctttgggtatgaattaggtcaggtggtccaacaagtgaaaaacccgggtaaaatcaaagccgaagaattagccggcttgttaaactctaccacaacaaacaacaccaatatcaatattgcaggcacaggaggcaatgtcgccgggactttgggcaacctttttatgaaccaattaggcaatttgattgatttgtatcccactttgaacactagtaatatcacacaatgtggcactactaatagtggtagtagtagtagtggtggtggtgcggccacagccgctgctactactagcaataagccttgtttccaaggtaacctggatctttatagaaaaatggttgactctatcaaaactttgagtcaaaacatcagcaagaatatctttcaaggcaacaacaacaccacgagccaaaatctctccaaccagctcagtgagcttaacaccgctagcgtttatttgacttacatgaactcgttcttaaacgccaataaccaagcgggtgggatttttcaaaacaacactaatcaagcttatggaaatggggttaccgctcaacaaatcgcttatatcctaaagcaagcttcaatcactatggggccaagcggtgatagcggtgctgccgcagcgtttttggatgccgctttagcgcaacatgttttcaactccgctaacgccgggaacgatttgagcgctaaggaattcactagcttggtgcaaaatatcgtcaataattctcaaaacgctttaacgctagccaacaacgctaacatcagcaattcaacaggctatcaagtgagctatggcgggaatattgatcaagcgcgatctacccaactattaaacaacaccacaaacactttggctaaagttagcgctttgaataacgagcttaaagctaacccatggcttgggaattttgccgccggtaacagctctcaagtgaatgcgtttaacgggtttatcactaaaatcggttacaagcaattctttggggaaaacaagaatgtgggcttacgctactacggcttcttcagctataacggcgcgggcgtgggtaatggccctacttacaatcaagtcaatttgctcacttatggggtggggactgatgtgctttacaatgtgtttagccgctcttttggtagtaggagtcttaatgcgggcttctttggggggatccaactcgcaggggatacttacatcagcacgctaagaaacagctctcagcttgcgagcagacctacagcgacgaaattccaattcttgtttgatgtgggcttacgcatgaactttggtatcttgaaaaaagacttgaaaagccataaccagcattctatagaaatcggtgtgcaaatccctacgatttacaacacttactataaagctggcggtgctgaagtgaaatacttccgcccttatagcgtgtattgggtctatggctacgccttctaa

SEQ ID NO: 2 - protein, 518 aa, Helicobacter pylori
MTQSQKVRFLAPLSLALSLSFNPVGAEEDGGFMTFGYELGQVVQQVKNPGKIKAEELAGLLNSTTTNNTNINIAGTGGNVAGTLGNLFMNQLGNLIDLYPTLNTSNITQCGTTNSGSSSSGGGAATAAATTSNKPCFQGNLDLYRKMVDSIKTLSQNISKNIFQGNNNTTSQNLSNQLSELNTASVYLTYMNSFLNANNQAGGIFQNNTNQAYGNGVTAQQIAYILKQASITMGPSGDSGAAAAFLDAALAQHVFNSANAGNDLSAKEFTSLVQNIVNNSQNALTLANNANISNSTGYQVSYGGNIDQARSTQLLNNTTNTLAKVSALNNELKANPWLGNFAAGNSSQVNAFNGFITKIGYKQFFGENKNVGLRYYGFFSYNGAGVGNGPTYNQVNLLTYGVGTDVLYNVFSRSFGSRSLNAGFFGGIQLAGDTYISTLRNSSQLASRPTATKFQFLFDVGLRMNFGILKKDLKSHNQHSIEIGVQIPTIYNTYYKAGGAEVKYFRPYSVYWVYGYAF

SEQ ID NO: 3 - DNA, 1557 bp, Helicobacter pylori
  CDS (1)..(1554)
atgataaaaaagaatagaacgctgtttcttagtctagccctttgcgctagcataagttatgccgaagatgatggagggtttttcaccgtcggttatcagctcgggcaagtcatgcaagatgtccaaaacccaggcggcgctaaaagcgacgaactcgccagagagcttaacgctgatgtaacgaacaacattttaaacaacaacaccggaggcaacatcgcaggggcgttgagtaacgctttctcccaatacctttattcgcttttaggggcttaccccacaaaactcaatggtagcgatgtgtctgcgaacgctcttttaagtggtgcggtaggctctgggacttgtgcggctgcagggacggctggtggcacttctcttaacactcaaagcacttgcaccgttgcgggctattactggctccctagcttgactgacaggattttaagcacgatcggcagccagactaactacggcacgaacaccaatttccccaacatgcaacaacagctcacctacttgaatgcggggaatgtgttttttaatgcgatgaataaggctttagagaataagaatggaactagtagtgctagtggaactagtggtgcgactggttcagatggtcaaacttactccacacaagctatccaataccttcaaggccaacaaaatatcttaaataacgcagcgaacttgctcaagcaagatgaattgctcttagaagctttcaactctgccgtagccgccaacattgggaataaggaattcaattcagccgcttttacaggtttggtgcaaggcattattgatcaatctcaagcggtttataacgagctcactaaaaacaccattagcgggagtgcggttattagcgctgggataaactccaaccaagctaacgctgtgcaagggcgcgctagtcagctccctaacgctctttataacgcgcaagtaactttggataaaatcaatgcgctcaataatcaagtgagaagcatgccttacttgccccaattcagagccgggaacagccgttcaacgaatattttaaacgggttttacaccaaaataggctataagcaattcttcgggaagaaaaggaatatcggtttgcgctattatggtttcttttcttataacggagcgagcgtgggctttagatccactcaaaataatgtagggttatacacttatggggtggggactgatgtgttgtataacatctttagccgctcctatcaaaaccgctctgtggatatgggcttttttagcggtatccaattagccggtgagaccttccaatccacgctcagagatgaccccaatgtgaaattgcatgggaaaatcaataacacgcacttccagttcctctttgacttcggtatgaggatgaacttcggtaagttggacgggaaatccaaccgccacaaccagcacacggtggaatttggcgtagtggtgcctacgatttataacacttattacaaatcagcagggactaccgtgaagtatttccgtccttatagcgtttattggtcttatgggtattcattctaa

SEQ ID NO: 4 - protein, 518 aa, Helicobacter pylori
MIKKNRTLFLSLALCASISYAEDDGGFFTVGYQLGQVMQDVQNPGGAKSDELARELNADVTNNILNNNTGGNIAGALSNAFSQYLYSLLGAYPTKLNGSDVSANALLSGAVGSGTCAAAGTAGGTSLNTQSTCTVAGYYWLPSLTDRILSTIGSQTNYGTNTNFPNMQQQLTYLNAGNVFFNAMNKALENKNGTSSASGTSGATGSDGQTYSTQAIQYLQGQQNILNNAANLLKQDELLLEAFNSAVAANIGNKEFNSAAFTGLVQGIIDQSQAVYNELTKNTISGSAVISAGINSNQANAVQGRASQLPNALYNAQVTLDKINALNNQVRSMPYLPQFRAGNSRSTNILNGFYTKIGYKQFFGKKRNIGLRYYGFFSYNGASVGFRSTQNNVGLYTYGVGTDVLYNIFSRSYQNRSVDMGFFSGIQLAGETFQSTLRDDPNVKLHGKINNTHFQFLFDFGMRMNFGKLDGKSNRHNQHTVEFGVVVPTIYNTYYKSAGTTVKYFRPYSVYWSYGYSF

SEQ ID NO: 5 - DNA, 656 bp, Helicobacter pylori
  CDS (567)..(656)
agatctatgaatctatgatatcaacactctttttgataaattttctcgaggtaccgagcttgaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccacaagctcggtaccgttgatcttcctatggtgcactctcagtacaatctgctctgatgcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcccagcgcttcgaacttctgatagacttcgaaattaatacgactcactatagggagaccacaacggtttccctctagaaataattttgtttaactttaagaaggagatatacatatgaaactgactcccaaagagttagacaagttgatgctccactacgctggagaattggctaaaaaacgcaaagaaaaaggcattaagctt

SEQ ID NO: 6 - protein, 30 aa, Helicobacter pylori
MKLTPKELDKLMLHYAGELAKKRKEKGIKL

SEQ ID NO: 7 - DNA, 656 bp, Helicobacter pylori
  CDS (567)..(656)
agatctatgaatctatgatatcaacactctttttgataaattttctcgaggtaccgagcttgaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccacaagctcggtaccgttgatcttcctatggtgcactctcagtacaatctgctctgatgcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcccagcgcttcgaccttctgatagacttcgaaattaatacgactcactatagggagaccacaacggtttccctctagaaataattttgtttaactttaagaaggagatatacatatgaaactgactcccaaagagttagacaggttgatgctccactacgctggagaattggctaaaaaacgcaaagaaaaaggcattaagctt

SEQ ID NO: 8 - DNA, 31 bp, Artificial Sequence
  misc_feature (1)..(31): Primer YZ019
ggaattccatatgaaactgactcccaaagag

SEQ ID NO: 9 - DNA, 27 bp, Artificial Sequence
  misc_feature (1)..(27): Primer RH132
ctgcagtcgactagaaaatgctaagag

(19) United States
(12) Patent Application Publication
(10) Pub. No.: US 2002/0161192 A1
(43) Pub. Date: Oct. 31, 2002

(54) Helicobacter pylori live vaccine

(76) Inventors: Thomas F. Meyer, Berlin (DE); Rainer Haas, Munchen (DE); Yan Zhengxin, Tubingen (DE); Oscar Gomez-Duarte, Tubingen (DE); Bernadette Lucas, Berlin (DE); Jochen Maurer, Stadtbergen (DE); Carol Patrice Gibbs, Augsburg (DE); Claus Tobias Lattemann, Neusaess (DE)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 09/976,297

(22) Filed: Oct. 15, 2001

Related U.S. Application Data
(63) Continuation-in-part of application No. 09/284,233, filed on Jul. 28, 1999.
Application No. 09/284,233 is a 371 of international application No. PCT/EP97/04744, filed on Sep. 1, 1997.

(30) Foreign Application Priority Data
Oct. 11, 1996 (EP) 96116337.5

(51) Int. Cl. C07K 1/00; C07K 14/00; C07K 17/00

(52) U.S. Cl. 530/350

(57) ABSTRACT

The present invention relates to novel recombinant live vaccines, which provide protective immunity against an infection by Helicobacter pylori and a method of screening H. pylori antigens for optimized vaccines.

The present invention relates to novel recombinant live vaccines, which provide protective immunity against an infection by Helicobacter pylori and a method of screening H. pylori antigens for optimized vaccines.

Helicobacter is a gram-negative bacterial pathogen associated with the development of gastritis, pepic ulceration and gastric carcinoma. Several Helicobacter species colonize the stomach, most notably H. pylori, H. heilmanii and H. felis. Although H. pylori is the species most commonly associated with human infection, H. heilmanii and H. felis also have been found to infect humans. High H. pylori infection rates are observed in third world countries, as well as in industrialized countries. Among all the virulence factors described in H. pylori, urease is known to be essential for colonisation of gnobiotic pigs and nude mice. Urease is an enzyme composed of two structural subunits (UreA and UreB). Previous studies have indicated that oral immunization using recombinant UreB plus cholera toxin were able to protect mice from gastric colonisation with H. felis and H. pylori (Michetti et al., Gastroenterology 107 (1994), 1002-1011). By oral administration of recombinant UreB antigens, however, in several cases only an incomplete protection can be obtained. Other H. pylori antigens shown to give partial protection are the 87 kD vacuolar cytotoxin VacA (Cover and Blaser, J. Biol. Chem. 267 (1992), 10570; Marchetti et al., Science 267 (1995), 1655) and the 13 and 58 kD heat shock proteins HspA and HspB (Ferrero et al., Proc. Natl. Acad. Sci. USA 92 (1995), 6499).

Attenuated pathogens, e.g. bacteria, such as Salmonella, are known to be efficient live vaccines. The first indications of the efficacy of attenuated Salmonella as good vaccine in humans came from studies using a chemically mutagenized Salmonella typhi Ty21a strain (Germanier and Furer, J. Infect. Dis. 141 (1975), 553-558), tested successfully in adult volunteers (Gilman et al., J. Infect. Dis. 136 (1977), 717-723) and later on in children in a large field trial in Egypt (Whadan et al., J. Infect. Dis. 145 (1982), 292-295). The orally administered Ty21a vaccine was able to protect 96% of the Egyptian children vaccinated during three years of surveillance. Since that time new attenuated Salomonella live vector vaccines have developed (Hone et al., Vaccine 9 (1991), 810-816), in which well defined mutations incorporated into the chromosome gave rise to non-virulent strains able to induce strong immune responses after oral administration (Tacket et al., Vaccine 10 (1992), 443-446 and Tacket et al., Infect. Immun. 60 (1992), 536-541). Other advantages of the live attenuated Salmonella vaccine include its safety, easy administration, long-time protection and no adverse reactions in comparison with the former inactivated wholesale typhoid vaccines (Levine et al., Typhoid Fever Vaccines, In; Plotkin S. A., Mortimer E. A. Jr. (eds.) Vaccines. Philadelphia; W B Saunders (1988), 333-361).

Mutants of S. typhimurium have been extensively used to deliver antigens because of the possibility to use mice as an animal model, which is believed to mimick S. typhi infections in humans. The attenuation of S. typhimurium most commonly used consists in site directed mutagenesis of genes affecting the synthesis of aromatic amino acids. Such strains, designated aro mutants, have a negligible pathogenicity, as demonstrated in animal models and human trials using these constructs (Hoiseth and Stocker, Nature 291 (1981), 238-239; Tacket et al. (1992), Supra). Advantage has been taken from the potent immunogenicity of live Salmonella vaccine to deliver heterologous antigens. Expression of specific antigens in attenuated Salmonella has conferred murine protection against several bacterial pathogens. The use of recombinant live vaccines, which are capable of expressing Helicobacter antigens and protecting the vaccinated animals, has not yet been described.

The use of attenuated live vaccines for the treatment of a Helicobacter infection has also not been rendered obvious. The reason therefor being that in the course of the Helicobacter infection a strong immune response against the pathogen per se is induced, which, however, does not lead to a protective immunity. Thus, it was highly surprising that a protective immune response is achieved when using recombinant attenuated bacterial cells as antigen carriers, which are capable of expressing a DNA molecule encoding a Helicobacter antigen. Apparently, recombinant attenuated bacterial cells expressing a Helicobacter antigen are capable of creating a qualitatively different immune response against the heterologous Helicobacter antigen than Helicobacter itself does against its own homologous antigen. Surprisingly, a non-protective immune response is thus transformed into an immune response protecting against Helicobacter infections. This unexpected observation renders it possible to use recombinant attenuated pathogens, e.g. bacterial cells, particularly Salmonella, as carriers for the screening of protective antigens, to apply the protective antigens identified in this manner in any vaccine against Helicobacter infections, and to use recombinant attenuated bacteria as carriers of protective antigens for the immunization against Helicobacter infections in humans and other mammals.

Thus, a subject matter of the present invention is a recombinant attenuated pathogen, which comprises at least one heterologous nucleic acid molecule encoding a Helicobacter antigen, wherein said pathogen is capable to express said nucleic acid molecule or capable to cause the expression of said nucleic acid in a target cell. Preferably the nucleic acid molecule is a DNA molecule.

The attenuated pathogen is a microorganism strain which is able to cause infection and preferably effective immunological protection against the actual pathogen but is no longer pathogenic per se. The attenuated pathogen can be a bacterium, a virus, a fungus or a parasite. Preferably it is a bacterium, e.g. Salmonella, such as S. typhimurium or S. typhi, Vibrio cholerae (Mekalanos et al., Nature 306 (1 983), 551-557), Shigella Species such as S. flexneri (Sizemore et al., Science 270 (1995), 299-302; Mounier et al., EMBO J. 11 (1992), 1991-1 999), Listeria such as L. monocytogenes (Milon and Cossart, Trends in Microbiology 3 (1995), 451-453), Escherichia coli, Streptococcus, such as S. gordonii (Medaglini et al., Proc. Natl. Acad. Sci. USA 92 (1995) 6868-6872) or Mycobacterium, such as Bacille Calmette Guerin (Flynn, Cell. Mol. Biol. 40 Suppl. 1 (1994), 31-36). More preferably the pathogen is an attenuated enterobacterium such as Vibrio cholerae, Shigelia flexneri, Escherichia coli or Salmonella. Most preferably the attenuated pathogen is a Salmonella cell, e.g. a Salmonella aro mutant cell. The attenuated pathogen, however, can be a virus, e.g. an attenuated vaccinia virus, adenovirus or pox virus.

The nucleic acid molecule which is inserted into the pathogen codes for a Helicobacter antigen, preferably a H. felis, H. heilmanii or H. pylori antigen, more preferably a H. pylori antigen. The Helicobacter antigen can be a native Helicobacter polypeptide, an immunologically reactive fragment thereof, or an immunologically reactive variant of a native polypeptide or of a fragment thereof. Further, the Helicobacter antigen can be a protective carbohydrate or a peptide mimotope simulating the three-dimensional structure of a native Helicobacter antigen. Peptide mimotopes can be obtained from peptide libraries presented on the surface of bacterial cells (of, PCT/EP96/ 01130). Of course, the transformed cell can also contain several DNA molecules coding for different Helicobacter antigens.

The nucleic acid molecules coding for Heliobacter antigens may be located on an extrachromosomal vector, e.g. a plasmid, and/or integrated in the cellular chromosome of the pathogen. When the pathogen is used as a vaccine, chromosomal integration usually is preferred.

Attenuated bacteria can be used to transcribe and translate said nucleic acid molecule directly in the bacterial cell or to deliver said nucleic acid molecule to the infected target cell, such that the DNA molecule is transcribed and/or translated by the eukaryotic target cell machinery. This indirect bacterial vaccination procedure, termed here as genetic vaccination, has been successfully used with Shigella as a carrier (Sizemore, D. R., Branstrom, A. A. & Sadoff, J. C. (11995) Attenuated Shigella as a DNA delivery vehicle for DNA-mediated immunization. Science 270:299-302).

In a preferred embodiment of the present invention the Helicobacter antigen is urease, a urease subunit or an immunologically reactive variant or fragment thereof or a peptide mimotope thereof. In a further preferred embodiment of the present invention the Helicobacter antigen is a secretory polypeptide from Helicobacter, an immunologically reactive variant or fragment thereof or a peptide mimotope thereof. A process for identifying Helicobacter genes coding for such secretory polypeptides, and particularly for adhesins, has been disclosed in the international patent application PCT/EP96/02544, which is incorporated herein by reference. This process comprises

a) preparing a gene bank of H. pylori DNA in a host organism containing an inducible transposon coupled to a marker of secretory activity,

b) inducing the insertion of the transposon into the H. pylori DNA and

c) conducting a selection for clones containing a secretory gene by means of the marker, and optionally further

d) conducting a retransformation of H. pylori by means of the DNA of clones containing genes having secretory activity, wherein isogenic H. pylori mutant strains are produced by means of integrating the DNA into the chromosome, and

e) conducting a selection detecting adherence-deficient H. pylori mutant strains.

Suitable examples of antigens obtainable by the above process are selected from the group consisting of the antigens AlpA, AlpB, immunologically reactive variants or fragments thereof or peptide mimotopes thereof. The nucleic and amino acid sequences of the antigens AlpA and AlpB have been disclosed in the international patent applications PCT/EP96/02545 and PCT/EP96/04124, which are incorporated herein by reference. Further, the nucleic and amino acid sequences of AlpB are shown in SEQ ID NO. 1 and 2, and the nucleic and amino acid sequences of AlpA in SEQ ID NO 3 and 4.

It is also conceivable, however, that an intracellular antigen is used which can be presented on the surface, e.g. by autolytic release, and confers immunological protection.

The presentation of the Helicobacter antigens in the recombinant pathogen according to the invention can be accomplished in different ways. The antigen or the antigens can be synthesized in a constitutive, inducible or phase variable manner in the recombinant pathogen. Concerning the constitutive or inducible synthesis of the Helicobacter antigens known expression systems can be referred to, as have been described by Sambrook et al., Molecular Cloning, A Laboratory Manual (1989), Cold Spring Harbor Laboratory Press.

Particularly preferred the antigens are presented in a phase variable expression system. Such a phase variable expression system for the production and presentation of foreign antigens in hybrid live vaccines is disclosed in EP-B-0 565 548, which is herein incorporated by reference. In such a phase variable expression system the nucleic acid molecule encoding the Helicobacter antigen is under control of an expression signal, which is substantially inactive in the pathogen, and which is capable of being activated by a spontaneous reorganization caused by a nucleic acid, e.g. DNA reorganization mechanism in the pathogen, egg. a specific DNA inversion process, a specific DNA deletion process, a specific DNA replication process or a specific slipped-strand-mispairing mechanism.

A recombinant cell having a phase variable expression system is capable of forming two subpopulations A and B, wherein the division into said subpopulations occurs by spontaneous reorganization in the recombinant nucleic acid, wherein said subpopulation A is capable of infection and immunologically active per se, while subpopulation B, which is regenerated from subpopulation A, produces at least one heterologous Helicobacter antigen and acts immunologically with respect to said additional antigen.

The activation of the expression signal encoding the Helicobacter antigen can be directly accomplished by nucleic acid reorganization or, alternatively, indirectly accomplished by activation of a gene encoding a is protein which controls the expression of the gene encoding the Helicobacter antigen. The indirect activation represents a system which allows the production of the Helicobacter antigen via a cascade system, which can be realized e.g. in that the gene directly controlled by DNA reorganization codes for an RNA polymerase which is specific for the promoter preceding the Helicobacter gene, or a gene regulator which in another specific manner induces the expression of the Helicobacter gene. In an especially preferred embodiment of the present invention the expression signal for the gene encoding the Helicobacter antigen is a bacteriophage promoter, e.g. a T3, T7 or SP6 promoter, and the activation of the expression signal is caused by a nucleic acid reorganization resulting in the production of a corresponding bacteriophage RNA polymerase in the pathogen,

The phase variable expression system can be adjusted to provide a preselected expression level of the Helicobacter antigen. This can be accomplished e.g. by modifying the nucleotide sequence of the expression signal, which is activated by the nucleic acid reorganization mechanism, and/or by inserting further genetic regulation elements.

The Helicobacter antigens can be produced in an intracellular, as well as in an extracellular manner in the pathogen according to the invention. For instance, autotransporter systems such as the IgA-protease system (cf. for instance EP-A-0 254 090) or the E. coli AIDA-1 adhesin system (Benz et al., Mol. Microbiol. 6 (1992), 1539) are suited as extracellular secretory system. Other suitable outer membrane transporter systems are the RTX-toxin transporters, e.g. the E. coli hemolysin transport system (Hess et al., Proc. Natl. Acad. Sci. USA 93 (1996), 11458-11483).

The pathogen according to the invention can contain a second heterologous nucleic acid, e.g. DNA molecule, which codes for an immunomodulatory polypeptide influencing the immune response quantitatively or qualitatively, apart from the nucleic acid molecule encoding the Helicobacter antigen. Examples of such immunomodulatory polypeptides are immune-stimulating peptides, cytokines like IL-2, IL-6 or IL-12, chemokines, toxins, such as cholera toxin B or adhesins.

The present invention also refers to a pharmaceutical composition comprising as an active agent a recombinant attenuated pathogen as described above, optionally together with pharmaceutically acceptable diluents, carriers and adjuvants. Preferably, the composition is a living vaccine. The vaccination routes depend upon the choice of the vaccination vector. The administration may be achieved in a single dose or repeated at intervals. The appropriate dosage depends on various parameters such as the vaccinal vector itself, or the route of administration. Usually the dosage comprises about 10⁶ to 10¹² cells (CFU), preferably about 10⁸ to 10¹⁰ cells (CFU) per vaccination. Administration to a mucosal surface (e.g. ocular, intranasal, oral, gastric, intestinal, rectal, vaginal or urinary tract) or via the parenteral route (e.g. subcutaneous, intradermal, intramuscular, intravenous or intraperitoneal) might be chosen. A method for the preparation of the living vaccine comprises formulating the attenuated pathogen in a pharmaceutically effective amount with pharmaceutically acceptable diluents, carriers and/or adjuvants.

The pharmaceutical composition may be provided in any suitable form, e.g. a suspension in suitable liquid carrier, such as water or milk, a capsule, a tablet etc. In a preferred embodiment of the present invention the composition is a lyophilized product which is suspended in a liquid carrier prior to use.

Further, the present invention refers to a method for preparing a recombinant attenuated pathogen as defined above, comprising the steps of a) inserting a nucleic acid molecule encoding a Helicobacter antigen into an attenuated pathogen, wherein the recombinant pathogen, e.g. a transformed bacterial cell, is obtained, which is capable of expressing said nucleic acid molecule or is capable to cause expression of said nucleic acid molecule in a target cell and b) cultivating said recombinant attenuated pathogen under suitable conditions. If the pathogen is a bacterial cell, the nucleic acid molecule encoding the Helicobacter antigen can be located on an extra-chromosomal plasmid. It is, however, also possible to insert the nucleic acid molecule into the chromosome of the pathogen.

Furthermore, the present invention refers to a method for identifying Helicobacter antigens which raise a protective immune response in a mammalian host, comprising the steps of:

a) providing an expression gene bank of Helicobacter in an attenuated pathogen and b) screening the clones of the gene bank for the ability to confer a protective immunity against a Helicobacter infection in a mammalian host. Preferably, this identification process takes place in a phase variable expression system, rendering possible a stable expression of all of the Helicobacter antigens Recombinant clones can then be applied as “pools” for the oral immunization of test animals, such as mice. The potential of these clones as protective antigen is then determined via a challenge infection with Helicobacter, e.g. a mouse-adapted H. pylori strain. Thus, there is a possibility of directly selecting optimized H. pylori vaccine antigens.

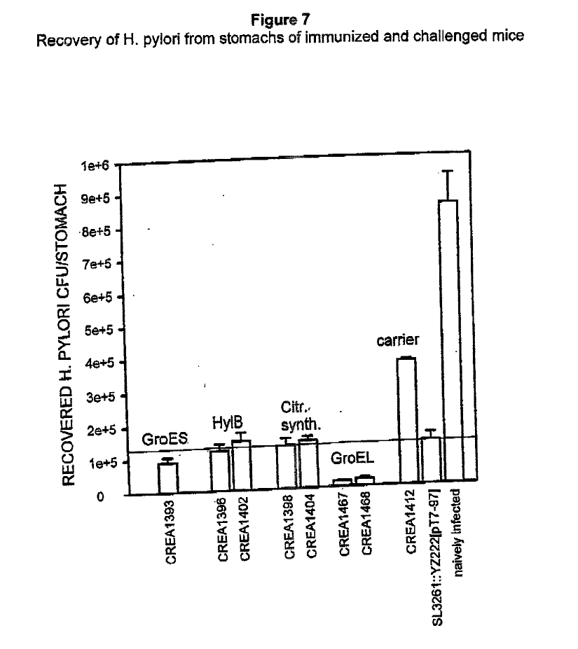

Based on the above disclosure new experiments with further immunogens are presented. It was confirmed that immunogens other than urease A/B can be employed for the manufacture of a live vaccine against Helicobacter. Herewith data are presented that an even higher level of protection compared to urease valued as the “golden standard” can be achieved (for comparison see FIG. 7) when using HylB, citrate synthase, GroEL and/or GroES as heterologous antigen in a microbial carrier, particularly a Salmonella carrier. Thus, a nucleic acid molecule encoding a HylB protein (such as disclosed in GenBank Accession Number AE000573), a citrate synthase (such as disclosed in GenBank Accession Number AE000525), a GroEL protein (such as disclosed in GenBank Accession Number AE000523) or a GroES protein (such as disclosed in GenBank Accession Number AE000523) or an immunologically active fragment or an epitope thereof is suitable for the manufacture of a live vaccine against a microbial infection, particularly a Helicobacter infection.

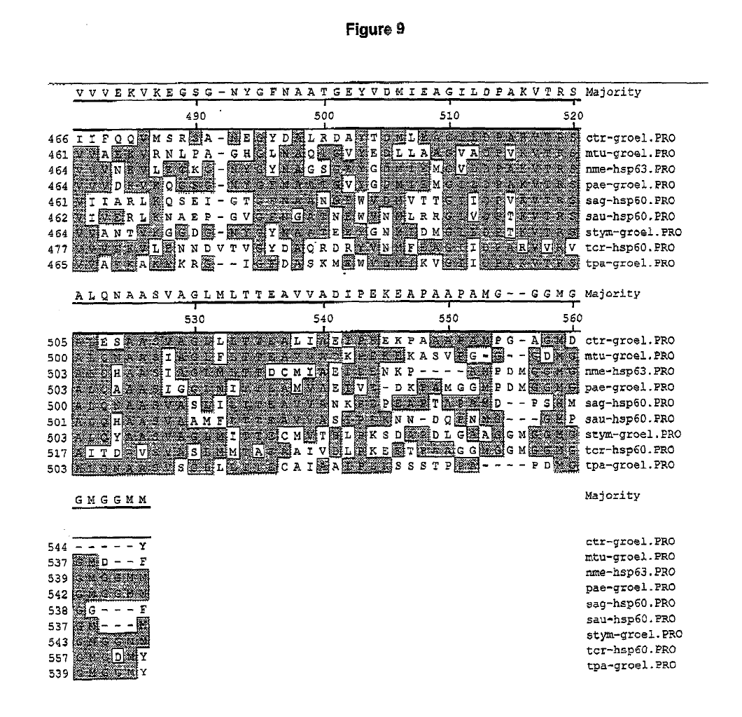

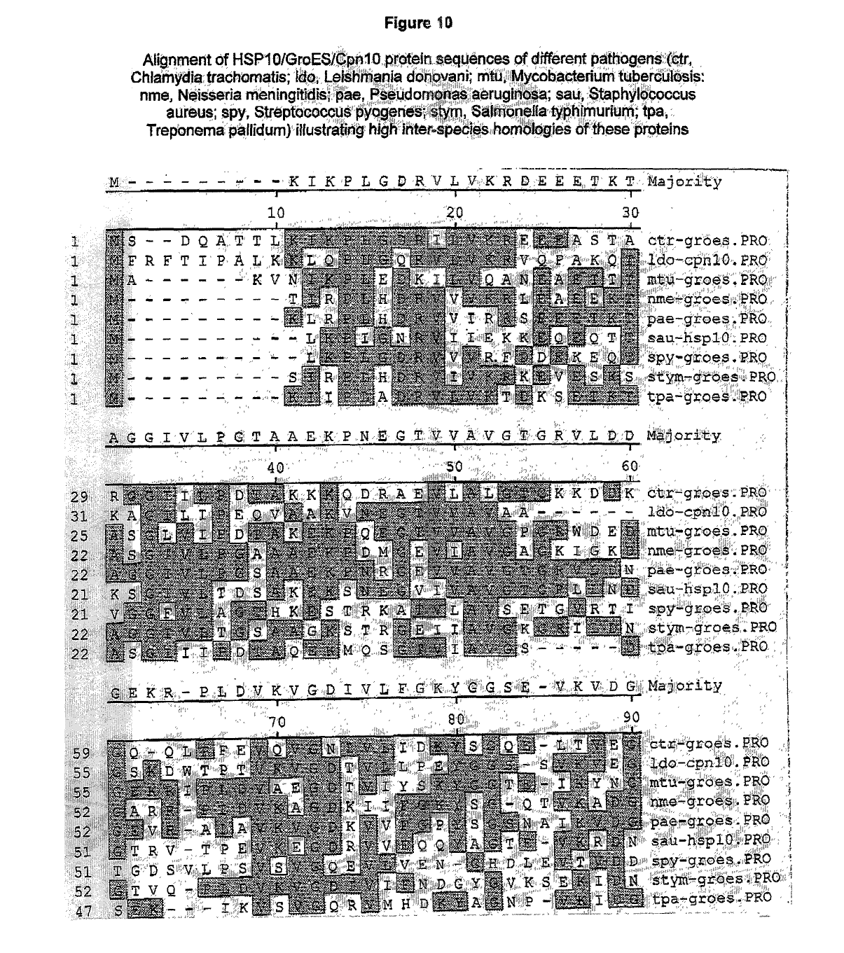

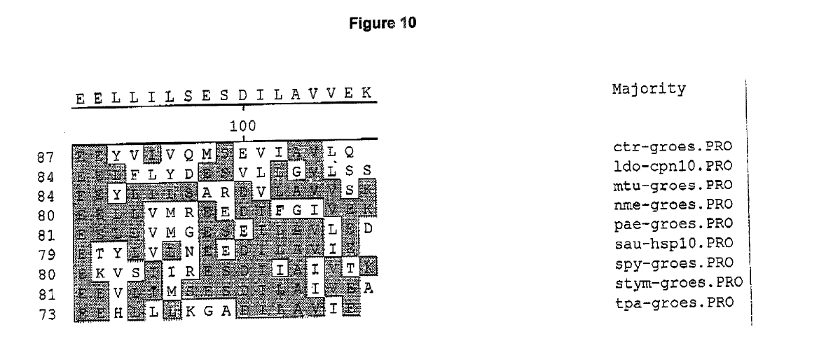

Further, the immunogens HylB, citrate synthase, GroEL and GroES can be used for the manufacture of a broad range live vaccine against microbial infections. This is due to their high homology between different microbial species which leads to cross protection from infections with one or several pathogenic microbial species (see e.g. FIG. 9 or 10). Thus, in addition to the respective immunogens from Helicobacter, the immunogens may be derived from other microbial species. The homology on the amino acid level (i.e. the percentage of identical amino acids) to the respective Helicobacter proteins may be at least 30%, 40%, 50%, 60%, 70%, 80%, 90% or even 95%, in increasing order of preference.

The nucleic acid sequence coding for said immunogens HylB, citrate synthase, GroEL or GroES can be derived from a bacterium such as Helicobacter spec., Shigella spec., Neisseria spec., Staphylococcus spec., Streptococcus spec., Pneumococcus spec., Pseudomonas spec., Treponema spec., Chlamydia spec., Mycobacterium spec., Bordetella spec., Clostridium spec., Salmonella spec., Campylobacter spec., Francisella spec., Coxiella spec., Haemophilus spec., Enterococcus spec., Enterobacter spec., Pasteurella spec., Vibrio spec., Klebsiella spec., Bartonella spec., Escherichia spec., Serratia spec., Bacillus spec., Legionella spec., Erwinia spec., Rickettsia spec., or Yersinia spec. However, microbial organisms other than bacteria such as Leishmania spec., Plasmodium spec., Trypanosoma spec or Amoeba spec. might be suitable as well.

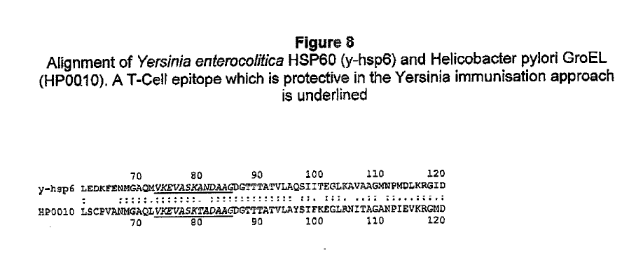

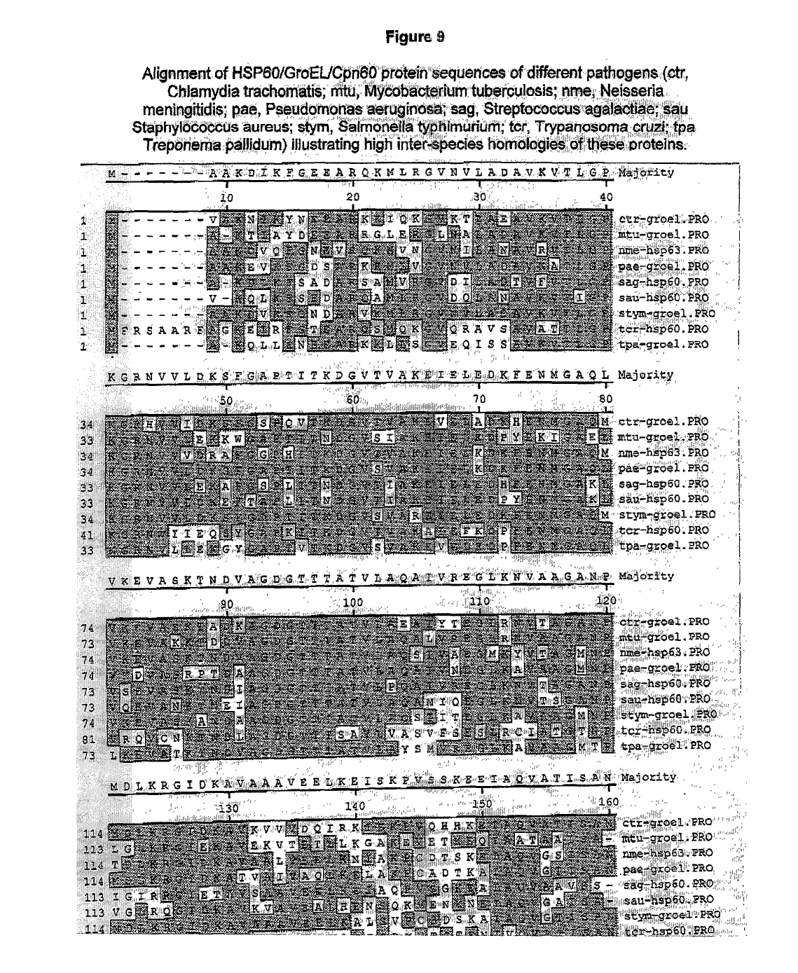

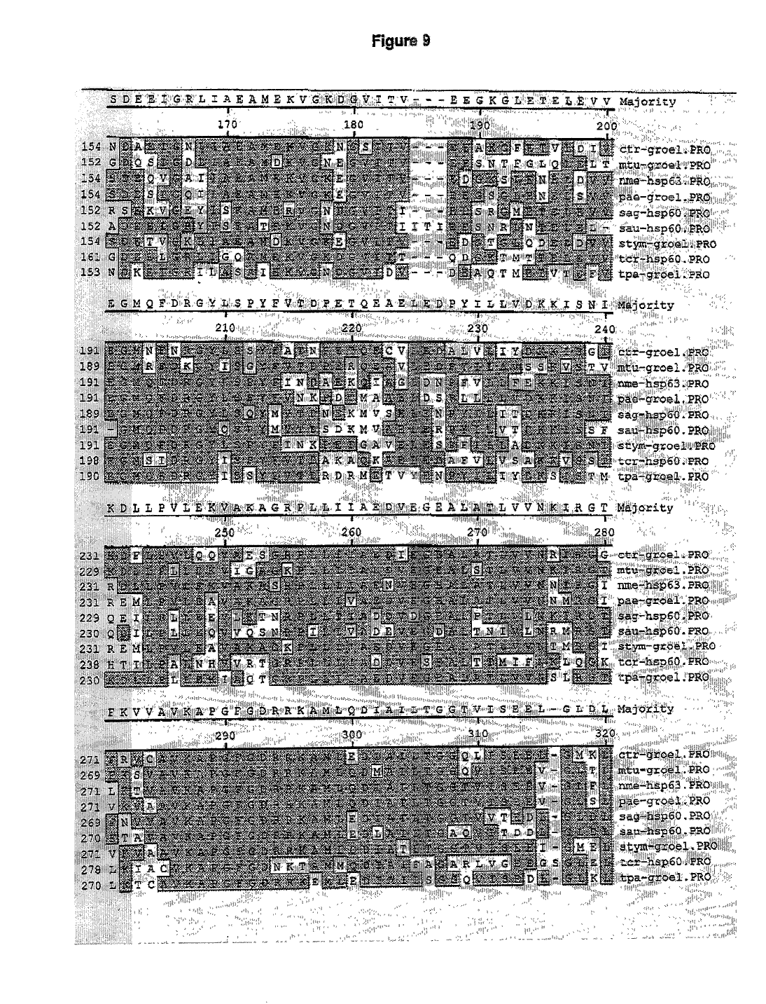

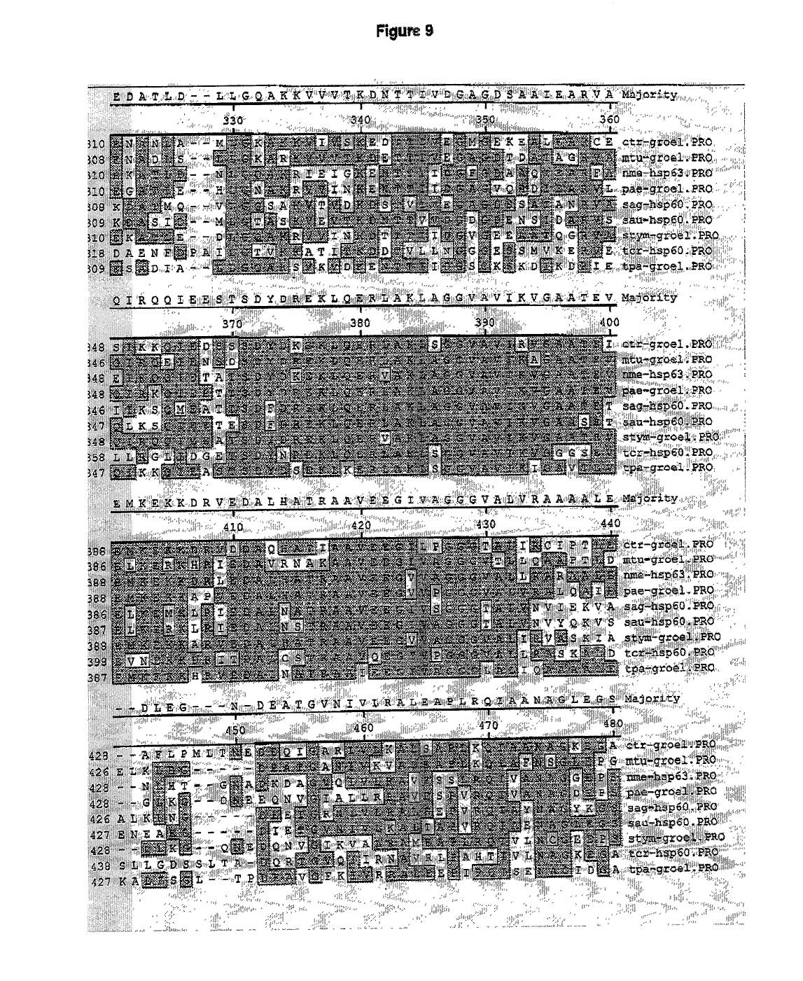

Instead of whole proteins also portions thereof such as immunogenic fragments, epitopes or clusters of epitopes can be used for the manufacture of said live vaccine. A selected T-cell epitope which is suitable for obtaining cross protection between Helicobacter and Yersinia is depicted in FIG. 8. T-cell epitopes typically comprise 8-15 amino acids in length but may vary in a length range from at least 5 amino acids to about maximally 20 amino acids. Further T-cell epitopes can be predicted from databases using i.e. the SYFPEITHI algorithm (Rammnensee, H., J. Bachmann, N. P. Emmerich, O. A. Barhor, and S. Stevanovic. 1999. SYFPEITHI: database for MHC ligands and peptide motifs. Immunogenetics. 50(3-4):213-9), (http://www.uni-tuebingen.de/uni/kxi/). Furthermore, surface exposure and protein hydrophilicity as means of predicting antigenicity, and thus B-cell epitopes may be characterized using algorithms as described by Hopp and Woods (Hopp, T. P., and K. R. Woods. 1983. A computer program for predicting protein antigenic determinants. Mol Immunol. 20(4):483-9) or by Janin and Wodak (Janin, J., and S. Wodak. 1987. Conformation of amino acid side-chains in proteins. J Mol Biol. 125(3):357-86). Thus, epitopes preferably have a maximal length of 30, 20, 15, 10 or even only 5 amino acids and even more preferably between 5 and 20 and most preferably between 8 and 15 amino acids. Preferably, the use of overlapping epitopes between human and microbial immunogens such as GroEL or GroES is avoided

GroES and GroEL proteins are ATPases and chaperons necessary for protein folding, unfolding, multimeric protein complex assembly, and disassembly. High degrees of homology are found throughout a wide variety of organisms. Synonymously, the term HSP60 (heat shock protein 60 kDa)/HSP10 (heat shock protein 10 kDa) and Cpn60/Cpn10 (Chaperon 60 kDa/10 kDa) often are used.

Recombinant GroES and GroEL proteins (corresponding with HP0010 and HP0011) from Helicobacter pylori were successfully employed in a prophylactic vaccination approach in mice using purified proteins as subunit vaccines as described by Ferrero et al. 1995 (Ferrero, R. L., J. M. Thiberge, I. Kansau, N. Wuscher, M S Huerre, and A. Labigne. 1995. The GroES homolog of Helicobacter pylori confers protective immunity against mucosal infection in mice. Proc Natl Acad Sci USA. 92(14):6499-503).

Also in other infectious diseases, GroEL proofed useful as an antigen in subunit vaccination approaches. In a Yersinia model, GroEL mediates protective immunity. Further examinations of (Yersinia) GroEL revealed protective T-Cell epitopes in this model (Noll, A., N. Bucheler, E. Bohn, and I. B. Autenrieth. 1997. Microbial heat shock proteins as vaccine. Behring Inst Mitt(98):87-98).

Citrate synthases are strongly conserved proteins among many organisms with a central function in metabolism. A 50152 kDa protein homologous to citrate synthase (corresponding with HP0026) was purified from Helicobacter pylori and used in a intra-Peyer's -Patch-vaccination model in mice (Dunkley, M. L., S. J. Harris, R. J. McCoy, M. J. Musicka, F. M. Eyers, L. G. Beagley, P. J. Lumley, K. W. Beagley, and R. L. Clancy. 1999. Protection against Helicobacter pylori infection by intestinal immunisation with a 50/52-ka subunit protein, FEMS Immunolog Med Microbiol. 24(2):221-5). In this subunit vaccine-approach, the purified protein was applied together with incomplete Freund's adjuvant by injection under the serosa of Peyer's patches of the mice intestines.

Several proteins from Helicobacter pylori were shown to mediate protective immunity in a prophylactic murine vaccination model using recombinant proteins purified from Escherichia coli (Hooking, D., E. Webb, F. Radcliff, L. Rothel, S. Taylor, G. Pinczower, C. Kapouleas, H. Braley, A. Lee, and C. Doidge. 1999. Isolation of recombinant protective Helicobacter pylori antigens. Infect Immun. 67(9):4713-9). One of these proteins was identified as HylB protein (HP0599 homologue), a (secreted) hemolysin secretion protein precursor. Mice were prophylactically immunized orogastrically at 7-day intervals at days 0, 7, 14 and 21 with ˜369 μg of the purified recombinant protein plus 10 μg Cholera Toxin as an adjuvant in a subunit vaccination approach.

After immunisation, mice were protected from a challenge with Helicobacter pylori or Helicobacter felis in all models described above. However, all immunogens were employed as subunit vaccines, with bacterial toxins or incomplete Freund's adjuvant as adjuvants.

In contrast to this, the present invention relates to a live vaccine comprising a bacterial carrier capable of expressing a nucleic acid molecule encoding at least one of said immunogens, wherein the expression is accomplished in a sufficient amount to induce at least partial immunity against a microbial infection in a mammalian host.

The bacterial carrier is capable of expressing the nucleic acid molecule, i.e. it is operatively linked to a gene expression system such as an in vivo inducible gene expression system. This expression system can be either homologous or heterologous to the employed carrier.

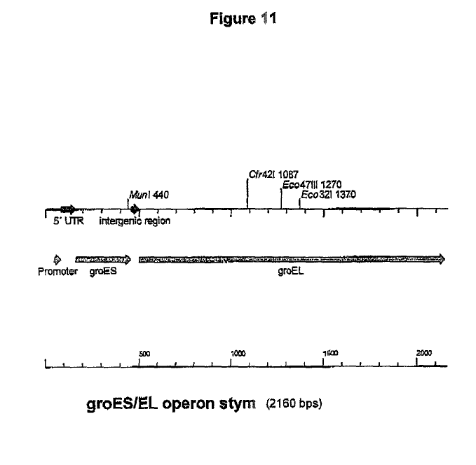

A preferred embodiment of an in vivo inducible expression system relates to the use of expression signals from the groES/EL operon. It is known (Buchmeier, N. A., and F. Heffron. 1990. Induction of Salmonella stress proteins upon infection of macrophages. Science. 248(4956):730-2) that expression of groES/EL in Salmonella is up-regulated as a consequence of interaction with the host, possibly due to the host's oxidative burst and other immunological events or fever. The operon structure of Salmonella typhimurium groES/EL is depicted in FIG. 11, (drawn from GenBank AS033231). Expression signals from the groELES operon comprise the promoter, a 5′ untranslated region, ribosome binding sites and/or the intergenic region between groES and groEL. Instead of or additionally to either groES or groEL gene sequence, any other nucleic acid, preferentially coding for an immunogen, can be introduced in said operon structure. This is accomplished by in-frame insertions, out-of-frame insertions, partial or complete substitution of groES or groEL and/or 3′ terminal appending of the nucleic acid coding for immunogens as described above. Thus, a further aspect of the invention relates to an expression system in a host cell for the expression of a nucleic acid molecule coding for an immunogen or fragment or epitope thereof heterologous to said host cell, comprising homologous or heterologous expression signals from the groES/EL operon and optionally a nucleic acid molecule coding for a GroES and/or GroEL protein.

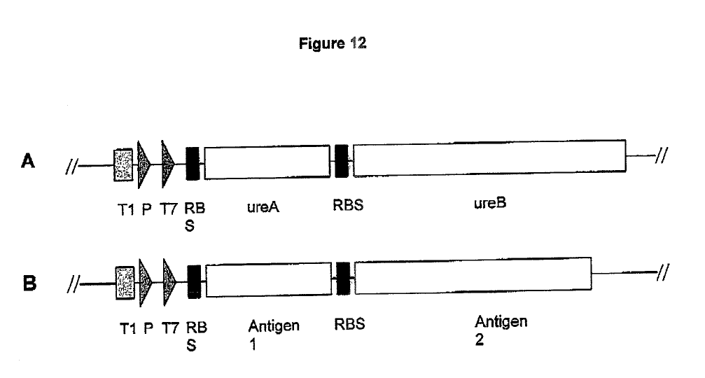

An additional preferred embodiment of an in vivo expression system is the use of expression signals from a ureAB operon e.g. as being provided on the plasmid pYZ97. Expression signals from the urea AB operon comprise a strong constitutive promoter, the 5′ untranslated region of the messenger RNA, the ribosome binding site of the ureA gene, the ribosome binding site of the ureB gene and/or the intergenic region between the coding sequences for ureA and ureB. The coding regions for ureA, ureB or ureA and ureB can be replaced with another immunogen resulting in expression of any combination of heterologous immunogens encoded on a plasmid carrying a recombinant ureAB operon (see FIG. 12).

Thus, an even further aspect the invention relates to an expression system in a host cell for the expression of a nucleic acid molecule coding for an immunogen or fragment or epitope thereof heterologous to said host cell, comprising expression signals from a Helicobacter UreaseA/B operon, preferably located on a suitable vector, e.g. the pYZ97 plasmid,

In a further preferred embodiment, said nucleic acid molecule is operatively linked to a two-phase gene expression system such as disclosed in WO92/11027, which is herein incorporated by reference.

The expression product of the nucleic acid molecule coding for an immunogen, particularly a HylB, a citrate synthase, GroEL or GroES protein may remain in the cytosol of said carrier, may be directed to the inner membrane, to the periplasm or outer membrane of said carrier, or may be secreted. In a more preferred embodiment the expression product is secreted by the type III or by the autotransporter system. Type III secretion is disclosed e.g. in WO98/53854 and an autotransporter system in WO97135022 which are herein incorporated by reference.

The nucleic acid molecule encoding a HylB, citrate synthase, GroEL or GroES protein or an immunogenic fragment or an epitope thereof can be homologous or heterologous to the employed carrier. For example, the nucleic acid molecule may be derived from an organism different from the carrier or the nucleic acid molecule may be derived from the carrier organism and being overexpressed in the carrier.

The immunogens can be expressed as single proteins or as proteins fused to an other immunogenic protein or fragment thereof wherein the immunogens can be either homologous or heterologous to the carrier as discussed above. In an even further preferred embodiment a nucleic acid molecule encoding a heterologous HylB, citrate synthase, GroEL or GroES protein or a fragment thereof is expressed as a fusion protein with a HylB, citrate synthase, GroEL or GroES protein homologous to the employed carrier organism or a fragment thereof, wherein a fusion protein containing heterologous and homologous portions is obtained.

In order to obtain a multivalent immunogen, several different immunogenic proteins can be expressed in the bacterial carrier. Thus, the invention also relates to a bacterial carrier capable of presenting a plurality of immunogens, particularly selected from HylB, citrate synthase, GroEL and GroES and optionally at least one further immunogen which is encoded by at least one further heterologous nucleic acid molecule. More preferably, the plurality of immunogens is selected from at least two proteins having immunogenic properties that induce at least partial protection against bacterial, viral or parasitic infection such as HylB, citrate synthase, GroES, GroEL (used in combination with each other), UreA, UreB, or catalase of H. pylori, listeriolysin and p60 of Listeria monocytogenes CFAI or CFAII of enterotoxigenic E. coli, F1 antigen of Yersinia pestis and others.

Enhanced immunogenicity can additionally be achieved by means of genetic fusions encoding an enzymatically active fusion partner protein or an immunomodulatory factor with said immunogen or a fragment thereof.

Thus, in an even more preferred embodiment, the multivalent immunogen is encoded by a nucleic acid molecule encoding a HylB, citrate synthase, GroES or GroEL protein or a fragment or an epitope thereof operatively linked to a further nucleic acid molecule encoding a further immunogen yielding a hybrid nucleic acid molecule encoding a fusion protein having at least two determinants. It should be noted that genetic fusions of three or more immunogenic components are also suitable.

To achieve efficient stimulation of the immune system the further nucleic acid molecule can be linked to the first nucleic acid encoding an immunogen in such a way that the resulting genetic fusion encodes a fusion protein of both determinants. Preferably, as outlined above, the immunogen confers—at least—partial protection against bacterial, viral or parasitic infection. Genetic fusions of three or more of said components are also suitable,

As described above, the use of said nucleic acid molecules for the manufacture of a live vaccine comprises the preparation of a bacterial carrier expressing one or more microbial immunogens. Thus, a further aspect relates to a recombinant carrier cell, which comprises at least one nucleic acid molecule encoding a HylB, a citrate synthase, a GroEL or a GroES protein or an immunologically active fragment or an epitope thereof, wherein said cell is capable of expressing said nucleic acid molecule and inducing protective immunity against a microbial infection in a mammalian host. The immunity may be induced against the carrier bacterium and a single pathogen from which the immunogen is derived such as Helicobacter but might also confer a broad range immunity against several microbial pathogens due to cross protection. In a preferred embodiment the bacterial carrier is an attenuated Salmonella cell.

In a preferred embodiment the recombinant carrier cell is capable of expressing a nucleic acid molecule encoding at least one immunogen and inducing protective immunity against to a Helicobacter infection in a mammalian host.

The nucleic acid molecule encoding the immunogen can be operatively linked to a gene expression system such as an in vivo inducible gene expression system and/or a two-phase gene expression system. The expression product of said nucleic acid molecule may remain in the cytosol of the carrier, may be directed to the inner membrane, to the periplasm or to the outer membrane of the carrier or may be secreted. As has been discussed above, the expression product is preferably secreted by the type III expression system or by an autotransporter system. The nucleic acid molecule can be homologous or heterologous to the recombinant carrier cell.

The recombinant carrier cell can express a fusion protein comprising homologous HylB, citrate synthase, GroEL or GroES protein or a fragment or an epitope thereof and a heterologous HylB, citrate synthase, GroES or GroEL protein or a fragment or an epitope thereof.

In a further embodiment, at least one further heterologous nucleic acid molecule can be expressed encoding an immunogen or a fragment or an epitope thereof. This fusion protein can comprise a HylB, citrate synthase, GroES or GroEL protein or a fragment or an epitope thereof with the expression product of the further nucleic acid molecule.

The carrier cell can be employed as a live vaccine against a microbial infection. Thus, in a further aspect, the invention relates to a pharmaceutical composition comprising as an active agent a recombinant carrier cell, e.g. a recombinant attenuated Salmonella cell as described above optionally together with a pharmaceutically acceptable diluent, carrier cell and/or adjuvant. Preferably, the vaccine is administered to a mucosal surface, e.g. by the oral route. Accordingly, a further embodiment relates to a composition which is a live vaccine, wherein said composition is in a form suitable for administration to a mucosal surface.

In a further aspect the invention relates to a method of inducing protective immunity against a microbial infection such as a Helicobacter infection in a mammalian host comprising administering to a mammalian host in need thereof an effective amount of the pharmaceutical composition.

Still a further aspect of the invention relates to a method of treating a microbial infection, comprising administering to a subject in need thereof the pharmaceutical composition comprising the attenuated Salmonella cell in a pharmaceutically effective amount for inducing protective immunity against an existing infection.

In an even further aspect the invention relates to a method of preventing a microbial infection, comprising administering to a subject in need thereof the pharmaceutical composition comprising the attenuated Salmonella cell in a pharmaceutically effective amount for inducing protective immunity against a future infection.

The invention will be further illustrated by the following figures and sequence listings.

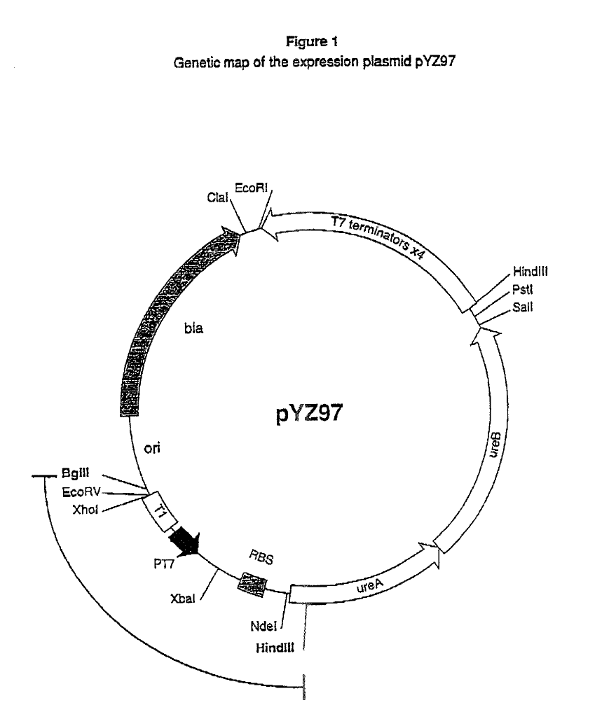

FIG. 1: shows a schematic illustration of the urease expression vector pYZ97, whereon the genes coding for the urease subunits UreA and UreB are located under transcriptional control of the T7 promoter φ10. There is a ribosomal binding site (RBS) between the T7 promoter and the urease genes. Further, the plasmid exhibits an origin of replication (ori), a β-lactamase resistance gene (bla) and 4 T7 terminators in series.

Apart from the expression by the T7 promoter, a constitutive low level expression of the urease A and B subunits can also be brought about via a cryptic promoter, which is located upstream from the T7 promoter, on the plasmid pYZ97.

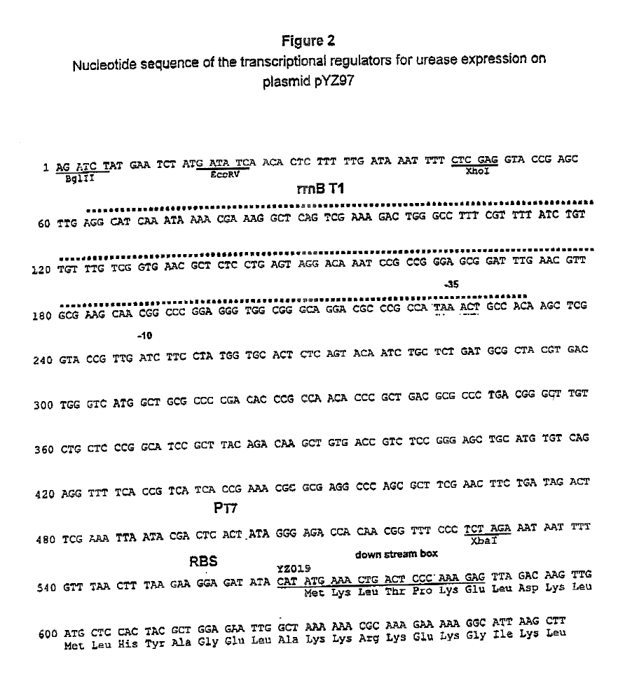

FIG. 2: shows the nucleotide sequence of the transcriptional regulation region for urease expression and the beginning of the amino acid sequence of urease subunit A on plasmid pYZ97.

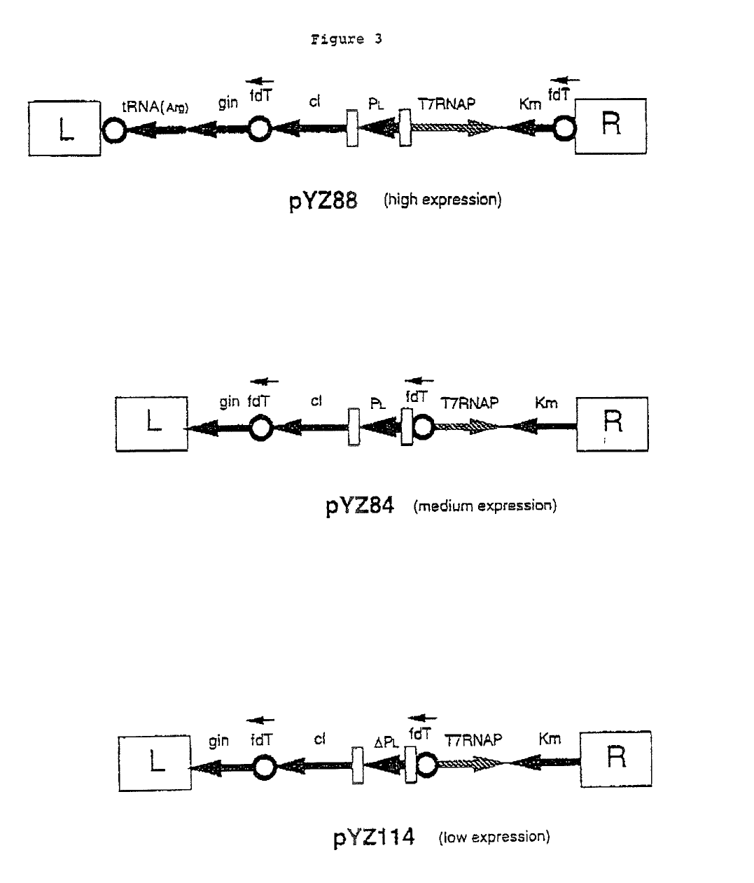

FIG. 3: shows a schematic illustration of the T7 RNA polymerase (T7RNAP) expression cassettes pYZ88, pYZ84 and pYZ114, which can be integrated into the chromosomes of bacteria.

In the high-expression cassette pYZ88 the lambda PL promoter is located in inverse orientation, upstream from the T7RNAP gene. A gene for the temperature-sensitive repressor cI 857 (cI) is under control of this promoter. A terminator of the bacteriophage fd (fdT) is situated upstream from the cl gene. The gin gene (Mertens, EMBO J. 3 (1984), 2415-2421) codes for a control enzyme of a DNA reorganization mechanism. A DNA sequence coding for the tRNA Arg is located downstream from the gin gene.

In phase A the PL promoter responsible for the expression of the T7RNAP gene is directed in the direction of the cI857 gene and the gin gene. The consequence of this is that an active repressor is formed at the permissive temperature of 28° C. and reduces the transcription from the PL promoter. At a higher temperature the transcription of the PL promoter is increased, since the repressor is inactivated at least partially under such external influences. The temperature-dependent increase in the transcription also causes a corresponding increase in the expression of the following gin gene, which as a control enzyme catalyses the inversion of the PL promoter and the transition in phase B, in which the T7RNAP gene is expressed.

In the high-expression system pYZ88 a further fdT transcription terminator is located between a kanamycin-resistance gene (km) and the promoter of this gene. In this manner, the synthesis of an anti-sense RNA, inversely orientated to the T7RNAP gene, which normally contributes to the reduction of the T7RNAP expression, is reduced. This results in a high expression of the T7RNAP.

In the medium-expression system pYZ84 a transcription terminator (fdT) is located between the PL promoter and the start of the T7RNAP gene. In this manner the expression of the T7RNAP mRNA is reduced. Additionally, the anti-sense RNA affects the T7RNAP translation. Therefore, only a medium expression occurs

In the low-expression system pYZ114 a deletion of 100 bp in PL is additionally introduced (A PL). In this manner the activity of the PL promoter is reduced to a high extent, which leads to a lower T7RNAP expression and thus to a reduction of the it 15 UreA/B gene expression. In this construct the effect of the cryptic promoter on pYZ97 is already observed.

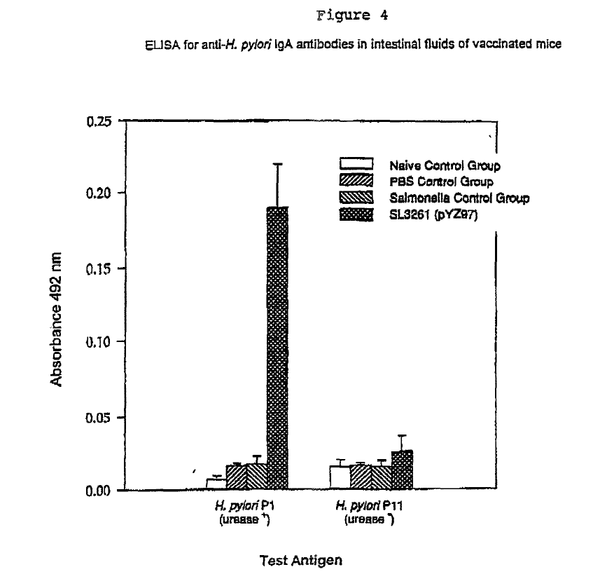

FIG. 4: shows the results of an ELISA for anti-H.pylori antibodies in intestinal fluids of vaccinated mice.

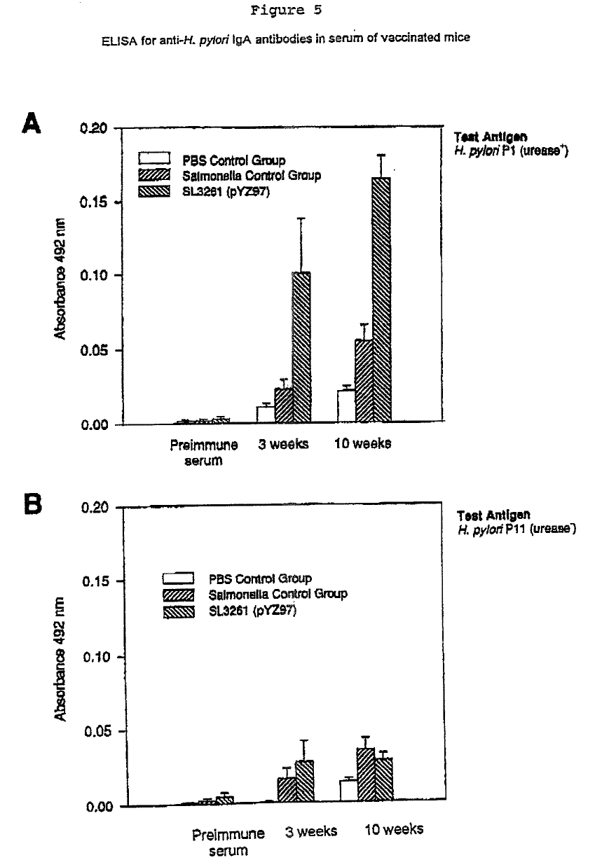

FIG. 5: shows the results of an ELISA for anti-H.pylori antibodies in the serum of vaccinated mice.

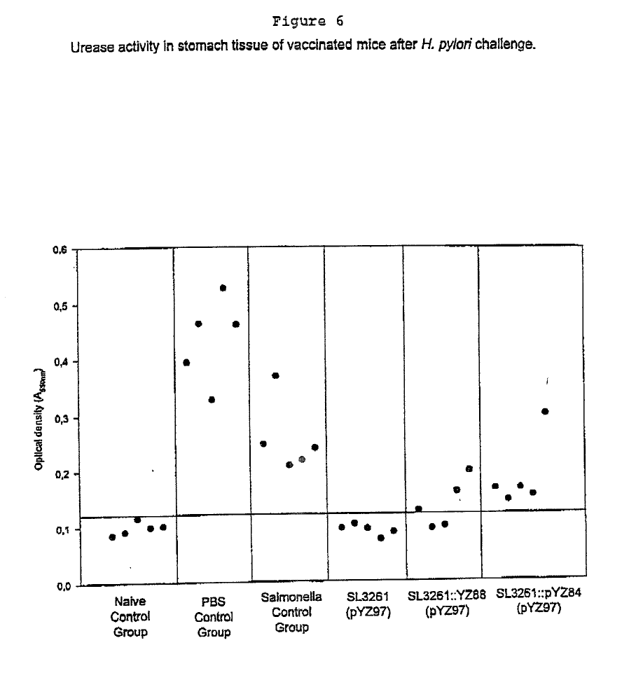

FIG. 6: shows the urease activity in the stomach tissue of vaccinated mice after H.pyroli challenge.

FIG. 7: shows the recovery of H. pylori from organisms of immunized and challenged mice.

FIG. 8: shows the alignment of a fragment of the Yersinia enterocolitica HSP60 (y-hsp60) and Helicobacter pylori GroEL (HP0010). A T-Cell epitope which is protective in the Yersinia immunisation approach is underlined.

FIG. 9: shows an alignment of HSP60/GroEL/Cpn60 protein sequences of different pathogens (ctr, Chlamydia trachomatis; mtu, Mycobacterium tuberculosis; nme, Neisseria meningitidis; pae, Pseudomonas aeruginosa; sag, Streptococcus agalactiae; sau Staphylococcus aureus; stym, Salmonella typhimurium; tcr, Trypanosoma cruzi; tpa, Treponema pallidum) illustrating high inter-species homologies of these proteins. Residues that match the consensus are boxed and shaded gray.

FIG. 10: shows an alignment of HSP10/GroES/Cpn10 protein sequences of different pathogens (ctr, Chlamydia trachomatis; ldo, Leishmania donovani: mtu, Mycobacterium tuberculosis; nme, Neisseria meningitidis; pae, Pseudomonas aeruginosa; sau, Staphylococcus aureus; spy, Streptococcus pyogenes; stym, Salmonella typhimurium; tpa, Treponema pallidum) illustrating high inter-species homologies of these proteins. Residues that match the consensus are boxed and shaded gray.

FIG. 11: shows the structure of the groES/EL operon from Salmonella typhimurium as found by Yamamoto et al. 1999 (unpublished, 6 GenBank accession number AB033231). The lower arrows depict the promoter and both genes, groES and groEL. The upper arrows represent the 5′ untranslated region and the intergenic region.

FIG. 12: shows in panel (A) the genetic structure of the synthetic ureAB operon and in panel (B) the genetic structure of a synthetic operon having ureA and ureB genes replaced by other antigens. T1: terminator rrnBT1; P: promoter; RBS: ribosome binding site of the respective open reading frame.

SEQ ID NO. 1 and 2 show the nucleotide sequence of the adhesin gene AlpB from H. pylori and the amino acid sequence of the polypeptide coded therefrom.

SEQ ID NO. 3 and 4 show the nucleotide sequence of the adhesin gene AlpA from H. pylori and the amino acid sequence of the protein coded therefrom.

SEQ ID NO. 5 and 6 show the nucleotide sequence of the transcriptional regulation region for urease expression and the beginning of the amino acid sequence of urease subunit A on plasmid pYZ97.

Experimental Part

### EXAMPLE 1

Cloning of the UreA and UreB Genes.

The structural genes encoding the urease, ureA and ureB, have been genetically cloned from chromosomal DNA of a clinical specimen P1 (formerly 69A) isolated at the University of Amsterdam and provided by Dr. Jos van Putten. The genes were isolated by a PCR-approach using the primer pair YZO19 (5′-GGAATTCCATATGAAACTGACTCCCAAAGAG-3′) and RH132 (5′-CTGCAGTCGACTAGAAAATGCTAAAGAG-3′) for amplification. The sequence of the primers was deduced from GenBank (accession numbers M60398, X57132). The DNA sequence of primer YZ019 covered the nucleotides 2659-2679 of the published sequence and further contained a translational regulatory sequence (down stream box; Sprengart, M. L. et al., 1990, Nuc. Acid. Res. 18:1719-1723) and a cleavage site for NdeI. The DNA sequence of primer RH132 covered the nucleotides 5071-5088 of the published sequence and a cleavage site for SelI. The amplification product was 2.4 kbp in size comprising the complete coding region of ureA and ureB genes without the original transcriptional start and termination sequences from the Helicobacter chromosome. The purified PCR-fragment was digested with NdeI and SalI and inserted into the corresponding cloning sites of T7 expression plasmid pYZ57 to yield the plasmid pYZ97.

pYZ57 was originally derived from plasmid pT7-7, which was described by Tabor (1990, In Current Protocols in Molecular Biology, 16.2.1-16.2.11. Greene Publishing and Wiley-lnterscience, New York). Two terminator fragments were introduced into the pT7-7 backbone at different sites by the following strategy: (1) The tandem T7 terminators. A 2.2 kbp EcoRI/HindIII fragment was excised from pEP12 (Brunschwig & Darzins, 1992, Gene, 111:35-41) and the purified fragment ligated with predigested pBA (Mauer, J. et al., 1997, J. Bacteriol. 179:794-804). The ligation product was digested with HindIII and ClaI. The resulting 2.2 kbp HindIII/ClaI fragment was subsequently inserted into predigested pT7-7.(2) The T1 terminator. A 230 bp HpaI/NdeI-fragment was excised from plasmid pDS3EcoRV (provided by Dr. H. Bujard; ZMBH, Heidelberg). The fragment was then further treated with Klenow to generate blunt ends. The purified rrnBT1 fragment was inserted into the previous pT7-7 derivative, predigested with BglII and subsequently bluntended by Klenow treatment. FIG. 1 describes the completed vector pYZ97 used for the expression of the urease genes coding for urease subunits UreA and UreB in S. typhimurium. As indicated in FIG. 1, the urease genes can be controlled by the T7 promoter 010. The ribosome binding site (RBS) is located between the T7 promoter and the urease genes. Further, the plasmid exhibits an origin of replication (ori) and a β-lactamase resistance gene (bla).

Apart from the expression controled by the T7 promoter, a constitutive moderate level expression of the urease A and B subunits does occur from a promoter driven by Salmonella RNA polymerase. The promoter is located upstream from the T7 promoter, on the plasmid pYZ97. For detailed molecular analysis, the purified BglII/HindIII-fragment of pYZ97 was subcloned into the PCR-Script™ SK(+)kit (Stratagene) and subjected to DNA-sequencing. The sequence data confirmed the various elements in their completeness (see FIG. 2 and SEQ ID NO.5 and 6): part of the ureA gene, the down-stream box, the RBS, the T7 promoter and the T1 terminator (rrnBT1). The sequence analysis also disclosed the region aco between the T1 terminator region and the T7 promoter where the Salmonella RNA polymerase promoter is localised. The sequence data suggests a location of this constitutive promoter between nucleotides 222-245 which have been deduced from structural predictions (Lisser & Margalit, 1993, Nuc. Acid. Res. 21:1507-1516).

### EXAMPLE 2

Immunological Protection by Administration of Live Vaccine

Materials and Methods

Bacterial Strains:

S. typhimurium SL3261 live vector vaccine strain was used as a recipient for the recombinant H. pylori urease plasmid constructs. S. typhimurium SL3261 is an aroA transposon mutant derived from S. typhimurium SL1344 wild type strain. S. typhimurium SL3261 is a non-virulent strain that gives protection to mice against infection with wild type S. typhimurium after oral administration (Hoiseth and Stocker (1981) Supra). S. typhimurium SL3261 and derivatives thereof, which contain the urease expression plasmid pYZ97 (extrachromosomal) and the T7RNAP expression cassettes pYZ88, pYZ84 or pYZ114, respectively (integrated into the chromosome) are indicated in table 1. Luria broth or agar was used for bacterial growth at 28° C. H. pylori wild type strain grown at 37° C. on serum plates was used for the challenge experiments.

Immunization of Mice:

Four weeks Balb/c mice purchased from Interfauna (Tuttlingen, Germany) were adapted two weeks in an animal facility before being used for experimentation. 150 μl of blood was taken retroorbitally from all mice to obtain preimmune serum. Retroorbital bleedings were repeated from all immunized mice 1 week and 3 weeks after immunization.

Eight groups of 5 mice including controls were used in this study (table 2). Group A, the naive control group, was not immunized with Salmonella neither challenged with wild type H. pylori. The rest of the groups were all orally immunized. Group B, a negative control group, did not receive Salmonella and was challenged with H. pylori. Mice from groups C to G were immunized with Salmonella vaccine strains and challenged with H. pylori. The last group H received recombinant urease B in combination with cholera toxin and was also challenged.

Prior to immunizations mice were left overnight without solid food and 4 hours without water. 100 μl of 3% sodium bicarbonate were given orally using a stainless steel catheter tube to neutralize the stomach pH. Then mice from group B received 100 μl PBS and mice from groups C to G received 1.0×10¹⁰ CFU of Salmonella in a 100 μl volume. Mice from group H received four times 100 μl of a mixture of recombinant H. pylori UreaseB plus cholera toxin, one dose every week. After every immunization water and food were returned to the mice.

H. pylori Challenge:

Four weeks after the first oral immunization mice from groups B to H were challenged with H.pylori. Mice were left overnight without solid food and without water 4 hours prior to the challenge. 100 μl of 3% sodium bicarbonate were given orally to the mice using a stainless steel catheter tube, followed by an oral dose of 5.0×10⁹ CFU/ml of Helicobacter pylori. Water and food were returned to the mice after the challenge.

Collection of Blood and Tissues from Mice:

Twelve weeks after the first immunization the mice were left overnight without food and subsequently sacrificed for analysis of protection and immune response. The mice were anaesthetized with Metoxyfluorane for terminal cardiac bleeding and prior to sacrifice by cervical dislocation. Under aseptic conditions, spleen and stomach were carefully removed from each mouse and placed on ice in separate sterile containers until further processing. Large and small intestine were obtained for further isolation of the intestinal fluid.

Processing of Stomach and Measurement of Urease Activity:

The degree of H. pylori colonisation in the mouse stomach was measured by the presence of active urease in the tissue. The Jatrox-test (Röhm-Pharma GmbH, Weiterstadt, Germany) was used according to the suppliers' directions. Stomach mucosa was exposed and washed with PBS, half of the antral portion of the stomach was immediately placed inside an Eppendorf tube containing the substrate for measurement of urease activity. Absorbance at 550 nm was measured after tubes were incubated for 4 hours at room temperature. The rest of the stomach tissue was stored at −20° C. for further treatments. The urease activity values obtained from the stomach of naive mice, which did not undergo immunization or challenge, were used to create a base line to indicate the absence of H. pylori infection and therefore protection.

TABLE 1
UreA and UreB expressing S. typhimurium vaccine strains

| Strains | Urease Expression | Source |
|---|---|---|
| S. typhimurium SL3261 | Negative | Hoiseth and |
| | | Stocker |
| S. typhimurium | Constitutive | this study |
| SL3262 pYZ97 | Low | |
| S. typhimurium | High T7-induced ex- | this study |
| SL3261::pYZ788pYZ97 | pression | |
| S. typhimurium | Medium T7-induced | this study |
| SL3261::pYZ84pYZ97 | expression | |
| S. typhimurium | Low T7-induced | this study |
| SL3261::pYZ114pYZ97 | expression | |

TABLE 2
Mice groups used for immunization

| | | No. of oral |
|---|---|---|
| Group | Immunogen | immunizations |
| A | None | 0 |
| B | PBS oral immunization | 1 |
| C | S. typhimurium S3261 | 1 |
| D | S. typhimurium S3261 pYZ97 | 1 |
| E | S. typhimurium S3261::pYZ88pYZ97 | 1 |
| F | S. typhimurium S3261::pYZ84pYZ97 | 1 |
| G | S. typhimurium S3261::pYZ114pYZ97 | 1 |
| H | Urease B plus cholera toxin | 4 |

Results:

In the control mice (groups B and C) 100% infection with H. pylori was observed. In the mice immunized with recombinant attenuated pathogens (groups D, E, F. G) between 0% and 60% infection (100% to 40% protection) was observed. Immuno-protection did not correlate with humoral anti-UreA and UreB response, suggesting that, in addition to humoral immunity, cellular immunity is critical for protection against H. pylori infection. The results indicate that oral immunization of mice with UreA and UreB delivered by S. typhimurium attenuated strain is effective to induce high levels of protection against H. pylori colonisation.

In the mice immunized with recombinant urease B plus cholera toxin considerably higher levels of urease activity were observed under said experimental conditions than when administering the recombinant attenuated pathogens according to the invention.

The results of the urease test have been illustrated in table 3.

TABLE 3

| Group | Mouse | E_{550 nm, 4 h} | E_{1 h} − E_{control} | E_{coeff.}*³ | Dilution |
|---|---|---|---|---|---|
| A | 1 | 0.085 | −0.022 | −0.066 | 200 μl + 400 μl |
| A | 2 | 0.091 | −0.016 | −0.048 | 200 μl + 400 μl |
| A | 3 | 0.116 | 0.009 | 0.027 | 200 μl + 400 μl |
| A | 4 | 0.099 | −0.008 | −0.024 | 200 μl + 400 μl |
| A | 5 | 0.101 | −0.006 | −0.018 | 200 μl + 400 μl |
| Control | | 0.107 | 0 | 0 | 200 μl + 400 μl |
| B | 1 | 0.394 | 0.292 | 0.876 | 200 μl + 400 μl |
| B | 2 | 0.464 | 0.362 | 1.086 | 200 μl + 400 μl |
| B | 3 | 0.329 | 0.227 | 0.681 | 200 μl + 400 μl |
| B | 4 | 0.527 | 0.425 | 1.275 | 200 μl + 400 μl |
| B | 5 | 0.462 | 0.36 | 1.08 | 200 μl + 400 μl |
| Control | | 0.102 | 0 | 0 | 200 μl + 400 μl |
| C | 1 | 0.248 | 0.145 | 0.435 | 200 μl + 400 μl |
| C | 2 | 0.369 | 0.266 | 0.798 | 200 μl + 400 μl |
| C | 3 | 0.209 | 0.106 | 0.318 | 200 μl + 400 μl |
| C | 4 | 0.219 | 0.116 | 0.348 | 200 μl + 400 μl |
| C | 5 | 0.24 | 0.137 | 0.411 | 200 μl + 400 μl |
| Control | | 0.103 | 0 | 0 | 200 μl + 400 μl |
| D | 1 | 0.143 | 0.002 | 0.004 | 300 μl + 300 μl |
| D | 2 | 0.156 | 0.015 | 0.03 | 300 μl + 300 μl |
| D | 3 | 0.142 | 0.001 | 0.002 | 300 μl + 300 μl |
| D | 4 | 0.114 | −0.027 | −0.054 | 300 μl + 300 μl |
| D | 5 | 0.133 | −0.008 | −0.016 | 300 μl + 300 μl |
| Control | | 0.141 | 0 | 0 | 300 μl + 300 μl |
| E | 1 | 0.127 | 0.027 | 0.081 | 200 μl + 400 μl |
| E | 2 | 0.094 | −0.006 | −0.018 | 200 μl + 400 μl |
| E | 3 | 0.099 | −0.001 | −0.003 | 200 μl + 400 μl |
| E | 4 | 0.161 | 0.061 | 0.183 | 200 μl + 400 μl |
| E | 5 | 0.198 | 0.098 | 0.294 | 200 μl + 400 μl |
| Control | | 0.1 | 0 | 0 | 200 μl + 400 μl |
| F | 1 | 0.166 | 0.025 | 0.05 | 300 μl + 300 μl |
| F | 2 | 0.145 | 0.004 | 0.008 | 300 μl + 300 μl |
| F | 3 | 0.166 | 0.025 | 0.05 | 300 μl + 300 μl |
| F | 4 | 0.154 | 0.013 | 0.026 | 300 μl + 300 μl |
| F | 5 | 0.301 | 0.16 | 0.32 | 300 μl + 300 μl |
| Control | | 0.141 | 0 | 0 | 300 μl + 300 μl |
| G | 1 | 0.084 | −0.019 | −0.057 | 200 μl + 400 μl |
| G | 2 | 0.087 | −0.016 | −0.048 | 200 μl + 400 μl |
| G | 3 | 0.269 | 0.166 | 0.498 | 200 μl + 400 μl |
| G | 4 | 0.085 | −0.018 | −0.054 | 200 μl + 400 μl |
| G | 5 | 0.092 | −0.011 | −0.033 | 200 μl + 400 μl |
| Control | | 0.103 | 0 | 0 | 200 μl + 400 μl |
| H | 1 | 0.638 | 0.531 | 1.593 | 200 μl + 400 μl |
| H | 2 | 0.282 | 0.175 | 0.525 | 200 μl + 400 μl |
| H | 3 | 0.141 | 0.034 | 0.102 | 200 μl + 400 μl |
| H | 4 | 0.135 | 0.028 | 0.084 | 200 μl + 400 μl |
| H | 5 | 0.171 | 0.064 | 0.192 | 200 μl + 400 μl |
| Control | | 0.107 | 0 | 0 | 200 μl + 400 μl |

### EXAMPLE 3

Construction and Molecular Analysis of Various Recombinant S. typhimurium Strains Expressing UreA/UreB Subunits.

Description of the S. typhimurium Strains Used for Immunization Experiments.

S. typhimurium SL3261(pYZ97) (construct A):

S. typhimurium SL3261 live vaccine vector strain was used as a recipient for the recombinant urease plasmid construct pYZ97.

S. typhimurium SL3261::YZ Series (pYZ97) (construct B):

These carrier strains are a derivative of S. typhimurium SL3261 which has been equipped with the T7 RNA polymerase (T7RNAP) expression cassettes schematically presented in FIG. 3. These expression cassettes encode the gene for T7RNAP which is expressed in a 2-phase modus (ON/OFF) as disclosed in a previous invention of Yan et al. (“Two phase system for the production and presentation of foreign antigens in hybrid live vaccines”, PCT/EP91/02478). The cassette can be integrated into the chromosome of bacteria and provide the cell in ON-position with optimal amount of T7RNAP for activation of T7RNAP-dependent expression plasmids such as pYZ97.

The principle of the YZ84 cassette is an invertible lambda PL promoter placed on a fragment that is inverted by the phage Mu invertase Gin (Yan & Meyer, 1996, J. Biotechnol. 44:197-201). Dependent on the orientation of the PL promoter either the gin gene (OFF-position) or the T7RNAP gene (ON-position) is expressed. The following regulatory elements have been included in YZ84: (1) The temperature-sensitive cIₜₛ lambda repressor (cI) which represses the PL promoter at 28° C. and dissociates at 37° C. (2) The phage fd terminator (fdT) reduces expression of gin gene in order to achieve moderate inversion rates of the PL promoter on the invertible fragment.

The 2-phase expression system enables high expression rates of foreign antigens, such as the urease subunits A and B. It is well known that high expression rates of foreign antigens reduce viability of Salmonella carrier thus diminishing immune response and consequently the protective potential. It was shown that the 2-phase system has a natural competence to improve survival of recombinant Salmonella which express large amounts of foreign antigen. In construct B. expression of the ureA and ureB genes is mainly under the control of the strong T7 promoter resulting in high production of the urease subunits. If the T7RNAP expression cassette is in OFF-position and no T7RNAP is present, the ureA and ureB genes are constitutively expressed in moderate range by the Salmonella promoter.

Analysis of UreA/B Subunits Produced by the Various S. typhimurium Strains Used for Immunization Experiments.

Salmonella constructs A and 5 were first analyzed by SDS-polyacrylamide gels for expression of UreA and UreB. The recombinant strains were grown at 37° C. in liquid Luria Broth supplemented with 100 μg/ml Ampicillin starting from an over night culture. The bacteria were harvested at logarithmic growth phase by centrifugation and the cell pellet was resuspended in 10 mM Tris-HCl and 10 mM EDTA, (pH 8.0) and cell-density adjusted to standard A₅₉₀=1.0 in all probes. The bacterial suspension was mixed with the same volume of SDS-sample buffer (Sambrook, J. et al. 1989, Molecular cloning: a laboratory manual. 2^{nd} ed. Cold Spring Harbor Laboratory Press, Cold Spring Harbor, N.Y.) and boiled for 5 min. 20 μl of suspension were loaded onto two SDS-10% polyacrylamide gels; one of the gels was stained with Coomassie blue stain and the other was electroblotted onto a nitrocellulose membrane and further processed for immunoblotting. The nitrocellulose membrane carrying the transferred proteins was blocked for 45 min at room temperature in 10 (v/w) % non-tat milk Tris-buffer-saline (TBS) (TrisHCl 100 mM, NaCl 150 mM, pH 7.2). After three washes in TBS-0.05 (v/v)% Tween-20, a 1:2000 dilution of rabbit anti-UreB antibody (AK 201) in 5 (w/v)% non-fat milk-TBS was added to the strip and incubated overnight at 4° C. Serum was obtained from rabbit immunised with recombinant urease B subunit purified via affinity chromatography. The membrane was washed three times for 10 min with 0.05 (v/v)% Tween-20 in PBS, and further incubated in 5 (w/v)% non-fat milk-TBS with goat anti-rabbit IgG antibody horse radish peroxidase conjugate for 45 min at room temperature. After three washes with 0.2 (v/v)% Tween-20 as above, the membrane was developed using the ECL kit (Amersham, Germany) following the recommendations of the suppliers.

Construct A:

Proteins of 67 kDa and 30 kDa were observed in the Coomassie stained gel of the whole cell lysate of construct A (S. typhimurium strain SL3261(pYZ97); these sizes correlate very well with those of UreB and UreA, respectively. Such proteins were absent in the control lanes containing the S. typhimurium SL3261 strain. Immunoblot analysis of the same protein samples using a rabbit anti-UreB antibody confirmed the 67 kDa protein observed in the Coomassie stained gel as UreB. Expression of ureB from S. typhimurium strain SL3261(pYZ97) was also examined at different phases of growth by incubating at 37° C. for 2, 6 and 11 hours, respectively. Expression of ureB was observed in all phases of growth including in the stationary phase; although, higher expression was observed at early phases of growth. The results obtained with strain SL3261(pYZ97) indicate that UreA and UreB proteins are non-toxic for Salmonella and can be expressed at 37° C. at any phase of bacterial growth.

Construct B:

Similar analysis were performed with construct B. The comparison of both constructs in SDS-PAGE analysis reveals that construct B is the more efficient producer whilst construct A has moderate expression of ureA and ureB. In the course of bacterial growth of construct B, the expression of ureA and ureB is constantly high over a longer time period even without antibiotic selection. This confirms the exceptional productivity of construct B in comparison to construct A.

In summary, our data indicate that UreA and UreB from H. pylori can be expressed in S. typhimurium without causing adverse effects to the bacteria, and are, therefore, suitable for animal protection experiments when delivered by Salmonella carriers.

Plasmid-Stability

Plasmid stability is essential to assure stable expression of antigens coded by genes which have been cloned into such plasmids.

In Vitro Plasmid Stability.

The ampicillin resistance marker present on plasmid pYZ97 and absent in the plasmidless S. typhimurium strain SL3261 was used as an indicator of plasmid stability. S. typhimurium strain SL3261 was grown in LB liquid medium at 28° C. for up to 100 generations as described previously (Summers, D. K. and D. J. Sherrat. 1984. Cell. 36:1097-1103). Every ten generations, the number of ampicillin resistant CFU was determined from the total number of colony forming units (CFU) of Salmonella by plating equal number of bacterial dilutions on plain LB-agar plates and LB-agar plates supplemented with 100 μg/ml ampicillin.

Plasmid Stability In Vivo.

Plasmid stability in vivo was analyzed by examining total CFU and ampicillin resistant CFU from mice spleen, two and seven days after oral infection of mice with 5.0×10⁹ CFU of S. typhimurium SL3261(pYZ97). Mice were orally infected with Salmonella as described above. Two days and seven days after infection mice were sacrificed under metoxyfluorane anesthesia, and the spleen was removed aseptically for further processing. The spleen was dissected in small pieces in a petri dish, mixed with 1 ml ice-cold ddH₂O, and passed several times through a 18 gauge needle to suspend the spleen cells. The cell suspension was then plated on LB-agar plates with or without 100 μg/ml ampicillin. Plates were incubated at 37° C. overnight and colonies counted the next day.

Plasmid stability in vivo was analyzed after infecting mice with one oral dose of 5.0×10⁹ CFU of S. typhimurium SL3261(pYZ97). Mice spleens were taken two and seven days after infection, and plated on LB-agar plates for examination of total CFU and ampicillin resistant CFU. 2.0×10⁴ ampicillin resistant CFU were isolated from the spleens after 48 h (Table 4). The CFU counts decreased to 56 at 7 days after immunization, but again, all were ampicillin resistant. The data indicate that plasmid pYZ97 is stable in Salmonella under in vitro and in vivo conditions and is suitable for the evaluation of urease subunits as protective antigens against mouse stomach colonization by H. pylori. The low recovery of Salmonella strain SL3261 seven days after infection confirms the attenuation of this strain which allows its safe use for delivery of urease into mice.

TABLE 4
Recovery of S. typhimurium SL3261pYZ97 strain from mouse spleens and
evaluation of pYZ97 plasmid stability in vivo.

| Time after infection | Total CFU^{a} | Percentage of Amp¹ CFU^{b} |
|---|---|---|
| 2 days | 2.0 × 10⁴ | 100 |
| 7 days | 56 | 100 |

^{a}Number of CFU of S. typhimurium isolated on LB plates without antibiotics from the mouse spleens two and seven days after mice had been orally inoculated with 5.0 × 10⁹ CFU of S. typhimurium strain SL3261(pYZ97).
^{b}Percentage of ampicillin resistant CFU from the total No. of CFU isolated from mouse spleens.

TABLE 5
Examination of urease activity and streptomycin resistant H. pylori in
stomach antrum from mice immunized with UreA and UreB-expressing
Salmonella.

| Mice group | No. | Urease activity¹ | CFU^{b} |
|---|---|---|---|
| Naive Control Group | 5 | 0.058 ± 0.004 | 0 ± 0 |
| PBS Control Group | 5 | 0.427 ± 0.059 | 2.7 × 10³ ± 1.0 × 10³ |
| SL3261pYZ97^{c} | 5 | 0.057 ± 0.006 | 62.6 ± 97.3 |

^{a}Urease activity is a mean value ± standard deviation.
^{b}Determination of CFU of the streptomicin resistant H. pylori P76 strain was carried out by plating a section of antrum stomach on serum plates supplemented with 200 μg/ml of streptomycin. H. pylori were recognized based on colony morphology, urease activity, and light microscopy examination. Values correspond to CFU ± standard deviation.
^{c}Mice immunized with S. typhimurium SL3261(pYZ97) expressing ureA and ureB from H. pylori as described in Materials and Methods.

### EXAMPLE 4

Protection Experiments with the Various Recombinant S. typhimurium Strains Expressing UreA/UreB Subunits in H. pylori Mouse Model.

Description of the Helicobacter pylori Strains Used for the Experiments

Urease-deficient H. pylori P11 strain is a derivative of P1, generated by transposon shuttle mutagenesis using the TnMax5 mini-transposon as disclosed in the invention of Haas et al. (“Verfahren zur Identifizierung sekretorischer Gene aus Helicobacter pylori”; PCT/EP96/02544). Insertion of TnMax5 has been mapped at the 3′-end of the ureA gene resulting in a defect expression of ureA and ureB due to transcriptional coupling of both genes.

Mouse-adapted H. pylori P49 strain was originally established by Dr. J. G. Fox (MIT, Boston, Mass.) from a feline isolate. H. pylori P76 strain is a streptomycin-resistant derivative of P49 generated by homologous recombination with chromosomal DNA from streptomycin-resistant H. pylori strain NCTC11637 as described by P. Nedenskov-Sorensen (1990, J. Infect. Dis. 161: 365-366).

All H. pylori strains were grown at 37° C. in a microaerobic atmosphere (5% O₂, 85% N₂, and 10% CO₂) on serum plates (Odenbreit, S. et al. 1996. J. Bacteriol. 178:6960-6967) supplemented with 200 μg/ml of streptomycin when appropriate.

Prophylactic Immunization Experiments with Mice.

Immunization experiments were carried out to test the ability of UreA and B delivered by Salmonella to protect mice from stomach colonization by H. pylori. In total, 5 independent immunisation experiments have been performed. Each experiment consisted of 5 groups each with 5 mice: (1) naive control group was mice neither immunized With Salmonella nor challenged with wild type H. pylori P49 or the streptomycin resistant derivative strain P76; (2) PBS control group was non-immunized mice that received PBS and were challenged orally with H. pylori; (3) Salmonella control group was mice immunized with attenuated S. typhimurium SL3261 strain alone and challenged with H. pylori, and (5) the vaccine group was the mice immunized with appropriate recombinant S. typhimurium construct (A+B) expressing UreA and UreB and challenged with H. pylori.

Prior to immunizations, mice were left overnight without solid food and 4 hours without water. 100 μl of 3% sodium bicarbonate were given orally using a stainless steel catheter tube to neutralize the stomach pH. Immediately after stomach neutralization, mice from the PBS control group received 100 μl PBS, and mice from the Salmonella control group and Salmonella vaccine group, received 5.0×10⁹ CFU of S. typhimurium strain SL3261 and the various recombinant constructs, respectively, in a total volume of 100 μl. Water and food were returned to the mice after immunization.

Four weeks after the oral immunization, mice from the PBS control-, Salmonella control- and vaccine-grows were challenged with 1.0×10⁹ CFU of H. pylori. Mice were left overnight without solid food and without water 4 hours prior to the challenge. 100 μl of 3% sodium bicarbonate were given orally to mice using a stainless steel catheter tube, followed by an oral dose of 1.0×10⁹ CFU/ml of H. pylori strains P49 or P76. Water and food were returned to mice after challenge.

### EXAMPLE 5

Immunological analyses of protection experiments with the various recombinant S. typhimurium strains expressing ureA/ureB subunits in H. pylori mouse model

Collection of Blood and Intestinal Fluid From Mice for Serological Analyses.

Antibody responses were evaluated from all mice using serum and intestinal fluid. 150 /11 blood were collected retro-orbitally before immunization and three weeks after immunization, before Helicobacter infection. The final bleeding was carried out 11 weeks after Salmonella immunization (6 weeks after challenge infection) by terminal cardiac puncture under metoxyfluorane anesthesia. The small intestines were taken from mice at the end of experiment and processed as described before (Elson, C. O. et al 1984. J. Immunol. Meth. 67:101-108) with minor modifications. Briefly, the content of intestines was removed by passing 2 ml of 50 mM EDTA pH 7.5 (Riedel) containing 0.1 mg/ml Soybean trypsin inhibitor (Sigma), The volume was adjusted to 5 ml with 0.15 M NaCl. The samples were vortexed vigorously, centrifuged 10 min at 2,500 rpm (Heraeus, Germany), and supernatant supplemented with 50 μl of 100 mM phenylmethylsulfonylfluoride (PMSF) (Serva) in 95% ethanol, followed by centrifugation at 1 3,000rpm for 20 min at 4° C. (Hermes). Supernatants were supplemented with 50 μl of 100 mM PMSF and 50 μl of 2% sodium azide (Merck) and left on ice 15 min before addition of 250 μl of 7% bovine serum albumine (Blomol). The samples were frozen at −20° C. until further use.

Analysis of Anti-Urease Antibodies in Mouse Sera and Intestinal Mucosa by ELISA.

Oral immunization with Salmonella is known to elicit IgA antibody responses. The IgA response against urease subunits in mice immunized with S. typhimurium construct A +B and in control mice was assessed by ELISA. A soluble extract of H. pylori P1 and its urease-deficient mutant derivative strain P11 was prepared in phosphate-buffer-saline by sonicating five times with a sonifier (Branson, Danbury, Conn.) at 5 sec intervals (35% pulses) for 45 sec. This suspension was centrifuged at 13,000 rpm (Heraeus, Germany) for 10 min at 4° C. to remove intact cells. The supernatant was used as antigen after determination of the protein content using the BiaRad kit. 96-well microtiter plates (Nunc, Germany) were coated with 50 μl aliquot of 50 μg/ml of antigen in sodium carbonate-bicarbonate buffer pH 9.6 and incubated overnight at 4° C. The wells were blocked with 1.0 (w/v)% non-fat milk in Tris-buffer-saline (TBS) for 45 min at room temperature and washed three times with TBS-0.05% Tween-20. The assays, which were performed in triplicate, used 50 μl of serum or gut washing diluted 1:100 or 1:2 respectively in 0.5 (w/v)% non-fat milk-TBS added to the wells and left overnight at 4° C. The wells were then washed three times with TBS-0.05% Tween 20, and a 1:3000 dilution of a goat anti-mouse IgA horse-radish peroxidase-conjugate (Sigma) was added to all wells and incubated overnight at 4° C. The color reaction was developed by incubation at 37° C. for 30 min with an orthophenylendiamine substrate in sodium acetate buffer and hydrogen peroxide. The reaction was stopped with 10 N H₂SO₄ and the A₄₉₂ was determined in an ELISA reader (Digiscan, Asys Hitech GmbH, Austria).

Mucosal Antibodies:

(Construct A) Intestinal fluid was taken from each sacrificed mouse at the end of the experiment (six weeks after the H. pylori challenge) and tested for the presence of anti-urease antibodies by using total cell extracts of H. pylori wild type (P1) and urease deficient mutant strains (P11). As shown in FIG. 4, the IgA antibody response against the wild type H. pylori extract was around 10-fold higher in immunized mice versus non-immunized or naive mice. The mucosal IgA antibody response against the urease-deficient H. pylori mutant was very low in all groups of mice indicating that most of the intestinal IgA antibody response in immunized mice was directed against urease.

Serum Antibodies:

(Construct A) The levels of serum IgA antibodies against a wild type and an urease-deficient H. pylori were examined prior to immunization, 3 weeks after immunization (before challenge) and 10 weeks after immunization (6 weeks after challenge with H. pylori). As shown in FIG. 5 panel A, the levels of anti-wild type H. pylori antibodies in mice immunized with urease-expressing S. typhimurium construct A were ˜20-fold higher at three weeks and 34-fold higher ten weeks after immunization with respect to the pre-immune serum. The serum IgA antibody response against the urease-deficient H. pylori strain at 3 and 10 weeks was low in all groups of mice including the mice immunized with Salmonella construct A (FIG. 5, panel B), indicating that most of the 19A antibody response in immunized mice is directed against the urease subunits. Low serum antibody responses against wild type H. pylori were also observed at ten weeks in non-immunized mice suggesting that the H. pylori challenge given three weeks earlier was enough to induce a specific antibody response in these mice. The IgA response to wild type H. pylori in mice immunized for three weeks with S. typhimurium SL3261 (Salmonella control group) increased moderately, which may be explained by the presence of antigens in Salmonella that are able to induce cross-reacting antibodies against H. pylori. In contrast, the antibody response against the urease-deficient H. pylori strain in immunized mice was as low as the antibody response of non-immunized mice (FIG. 5, panel B). This result suggests that most of the antibody response observed in immunized mice was against urease. Low antibody response against the urease-negative mutant was observed in the 10 weeks sera from mice given PBS or immunized with S. typhimurium SL3261, suggesting that the antibody response observed is due to the specific immune response against the H. pylori antigens given to these mice three weeks earlier during challenge. A low antibody response against the urease-deficient H. pylori strain was observed at three weeks in mice immunized with Salmonella either expressing or not expressing urease, but was absent in the mice given PBS. This confirms the presence of cross-reacting epitopes between proteins from Salmonella and H. pylori, respectively. (Construct B): The serological analysis of mice immunized with the construct B series achieved similar results indicating that higher production of antigen by recombinant Salmonella does not significantly increase antibody response.

Analysis of Anti-Urease Antibodies in Mouse Sera by Immunoblotting.

Expression of UreA and UreB from S. typhimurium necessary for the induction of mice specific immune response against H. pylori was analyzed. Identification of in vivo expression of UreA and UreB was carried out by looking for anti-UreA and anti-UreB antibodies in serum of mice immunized with Salmonella construct A and control mice. H. pylori whole-cell antigens were prepared from the wild type H. pylori strain P1. Bacteria were recovered from 3 serum plates, resuspended in PBS, and harvested by 10 min centrifugation at 5,000 g. The cell pellet was resuspended in 10 mM Tris-HCl and 10 mM EDTA, (pH 8.0) and cell-density adjusted to standard A₅₉₀=1.0 in all probes. The bacterial suspension was mixed with same volume of SDS-sample buffer (Sambrook, 1989) and boiled for 5 min, 20 μl Pellet were loaded onto a SDS-10% polyacrylamide gel. The proteins were electro-blotted onto a nitrocellulose membrane and cut into strips which were blocked for 45 min at room temperature in 10 (v/w)% non-fat milk Tris-buffer-saline (TBS) (TrisHCl 100 mM, NaCl 150 mM, pH 7.2). After three washes in TBS-0.05 (v/v)% Tween-20, a 1:80 dilution of mouse serum in 5 (w/v)% non-fat milk-TBS was added to the strips and incubated overnight at 4PC. Sera was obtained from mice non-immunized and immunized with Salmonetta. After three washes, the strips were incubated with a goat anti-mouse IgG horse-radish peroxidase conjugate (Sigma) diluted 1:3000 in 5 (w/v)% non-fat milk-TBS. The ECL chemi-luminescence detection kit (Amersham, Germany) was used for development of blots according to the supplier's directions.

Serum from immunized and non-immunized mice was obtained 3 weeks after immunization prior to the challenge With H. pylori and tested against whole-cell lysates of the wild type H. pylori P1 strain expressing UreA and UreB. Proteins of 67 kDa and 30 kDa in size, corresponding to UreB and UreA, respectively, were recognized by serum from immunized mice immunized with construct A. These bands were not observed in strips tested with serum from non-immunized mice or mice immunized with Salmonella only, suggesting that urease expressed by the Salmonella vaccine strain was able to induce a specific antibody response against both UreA and UreB of a wild type H. pylori strain. Similar results were obtained with construct B.

### EXAMPLE 6

Determination of H. pylori Colonisation in Mice Pretreated with the Various Recombinant S. typhimurium Strains Expressing UreA/UreB Subunits in H. pylori Mouse Model

Processing of Stomach and Measurement of Urease Activity.

Urease-test:

Analysis of protection against stomach colonization by H. pylori was performed by testing for urease activity in the antral portion of the mouse stomach. Measurement of urease activity is a very reliable, sensitive and specific method to test for the presence of H. pylori infection (NIH consensus development on Helicobacter pylori in peptic ulcer disease. 1994. Helicobacter pylori in peptic ulcer disease. JAMA. 272:65) and is routinely used in clinical settings (Kawanishi, M., S. et al 1995. J. Gastroenterol. 30:16-20; Kamija, S. et al 1993, Eur. J. Epidemiol. 9:450-452; Conti-Nibali, S. et al 1990. Am. J. Gastroenterol. 85:1573-1575) and in animal research (Gottfried, M. R. et al 1990. Am. J. Gastroenterol. 85:813-818). The Jatroxtest (Röhm-Pharma GmbH, Weiterstadt, Germany) was used according to the suppliers directions. Eleven weeks after immunization with Salmonella, mice were sacrificed and the stomach was carefully removed under aseptic conditions. The stomach was placed in ice-cold PBS in an sterile container, and the mucosa was exposed by making an incision along the minor curvature with a sterile blade. The stomach was rinsed with PBS to remove food residues and dissected to isolate the antral region from the corpus region. Half of the antral portion of the stomach was immediately placed inside an Eppendorf tube containing 500 μl of the urease substrate from Jatrox-test. The stomach sample was incubated 4 h at room temperature and the absorbance at 550 nm (A₅₅₀) measured. The urease activity values obtained from the stomach of naive mice, which did not undergo immunization or challenge, were used to determine the baseline. The baseline corresponded to the average urease activity value from five naive mice stomachs tested plus two times the standard deviation of this average. Urease activity values higher than the baseline were considered H. pylori colonization positive and values below the baseline were considered H. pylori colonization negative.

Cultivation Experiment:

The left portion of the antral region of stomachs obtained from mice challenged with the streptomycin resistant H. pylori strain P76 were plated on serum plates supplemented with 200 μg/ml of streptomycin and incubated under standard conditions. After three days incubation, bacteria were identified as H. pylori based on colony morphology, microscopic examination, and urease activity. The number of colony forming units (CFU) of H. pylori grown on plates was determined from each mouse stomach sample.

Urease Test (Construct A Vs. B):

Mice immunized with ˜5.0×10² CFU of Salmonella and challenged with 1.0×10⁹ CFU of H. pylori strain P49, as well as control mice, were sacrificed under anesthesia and a section of the antral region of the stomach was taken for measurement of urease activity. As shown in FIG. 6, 100% of the mice immunized with UreA and B delivered by Salmonella construct A had urease activity below the baseline, indicating the absence of H. pylori colonisation. In contrast, 100% of the non-immunized mice (PBS) and the mice immunized with S. typhimurium strain SL3261 alone, had urease activity measurements far above the baseline indicating stomach colonization by H. pylori, The naive group of mice, which did not undergo immunization or challenge, was used to set so the baseline of urease activity.

Salmonella of the construct B-series had urease activity values above the baseline indicating stomach colonization by H. pylori challenge strain. However, the urease activities within this group were lower as in the controls suggesting a partial protection status of mice immunized with the Salmonella construct B series (FIG. 6). Both Salmonella constructs, A and B, mediate similar antibody response but differed in expression of ureA and ureB. We conclude from this that The quantity of expressed urease antigen is relevant to gain optimal protection.

Construct A:

To correlate stomach colonization by H. pylori with urease activity a new protection experiment was performed by immunizing mice orally with Salmonella construct A and challenging them with the streptomycin resistant H. pylori P76 strain. Urease activity values correlated with the number of CFU of H. pylori identified. In two of the five mice immunized with urease-expressing Salmonella, no H. pylori CFU were detected and the average number of CFU in all five immunized mice was only 62. In contrast, the number of CFU in non-immunized mice was 2,737, which corresponds to 44-fold higher colonization. These data indicate that mice immunized with uresase-expressing Salmonella were able to eliminate or significantly decrease colonizing H. pylori from mouse stomachs.

### EXAMPLE 7

Protection Experiments with Various Antigens and Expression Signals in the H. pylori Mouse Model

A live vaccine approach is used to protect against H. pylori infection. Recombinant Salmonella is employed as carrier of various H. pylori immunogenic proteins. Several bacterial constructs with differing antigens mediate protection against H. pylori challenge in a mouse model.

The following protocol was used for vaccination. On day 0, six to eight week old female specific pathogen free BALB/c mice were immunised intra-gastrically with 200 μl (100 mM NaHCO₃) of a bacteria suspension containing 10⁹ colony-forming units (cfu) of Salmonella typhimurium SL3261::YZ222 ΔthyA expressing the respective antigens. Antigens were coded on stabilised plasmids employing the plasmid-coded thyA gene as a means of balanced lethality to complement the chromosomally deleted thyA. Plasmids allowed for expression of the various antigens driven by one out of three respective expression signals (P_{phoP}, P_{nirB}, P_{T7}, Table 6). On day 28, mice were challenged intra-gastrically with a total of 10⁹ cfu of H. pylori strain P76 (strep^{R}) suspended in a volume of 100 μl (1 00 mM NaHCO₃). At day 49, mice were sacrificed, and half of the stomachs was plated on GC agar supplemented with 8% serum and 10 μg/ml streptomycin, allowing growth of H. pylori P76 (strep^{R}), and the number of streptomycin-resistant H. pylori cfu was determined (FIG. 1).

The live vaccination approach proved protective immunity using urease A and B subunits under control of a PT7 expression signal as a positive control. The respective plasmid was termed pYZ97 and described elsewhere (Gomez-Duarte et al., 1998). Protection was determined by cfu counting, and a nmock control construct (CREA1412) did not mediate protection. Naively infected mice also were highly susceptible to a H. pylori challenge.

In general, the results were independent of the expresssion signal used. As determined by cfu counting all 4 antigens employed, GroES (CREA1393), HylB (CREA1396 and CREA1402), citrate synthase homolog, (CREA1398 and CREA1404), and GroEL (strains CREA1467 and CREA1468) were protective. Thus, specific protection due to recombinant H. pylori antigens expressed by a live vector is achieved.

The Helicobacter immunogens employed raised protection levels similar or even better than Urease A and E subunits according to cfu counting.

TABLE 6
Combinations of expression signals and various antigens used for
vaccination

| Construct | Antigen | Expression signal |
|---|---|---|
| CREA1393 | GroES | P_{phoP} |
| CREA1396 | HyIB | P_{T7} |
| CREA1398 | citrate synthase | P_{T7} |
| | homolog | |
| CREA1402 | HyIB | P_{nirB} |
| CREA1404 | Citrate synthase | P_{nirB} |
| | homolog | |
| CREA1412 | none | none |
| CREA1467 | GroEL | P_{nirB} |
| CREA1468 | GroEL | P_{phoP} |
| SL3261::YZ222 | Urease A subunit | P_{T7} |
| ΔthyA (pT7-97) | Urease B subunit | internal promoter |

1. a recombinant attenuated microbial pathogen, which comprises at a least one heterologous nucleic acid molecule encoding a Helicobacter antigen, wherein said pathogen is capable to express said nucleic acid molecule or capable to cause the expression of said nucleic acid molecule in a target cell.

2. The pathogen according to claim 1, which is an enterobacterial cell, especially a Salmonella cell.

3. The pathogen according to claim 1 or 2, which is a Salmonella aro mutant cell.

4. The pathogen according to any of claims 1-3,
wherein the Helicobacter antigen is urease, a urease subunit, an immunologically reactive fragment thereof, or a peptide mimotope thereof.

5. The pathogen according to any one of claims 1-3,
wherein the Helicobacter antigen is a secretory polypeptide from Helicobacter, an immunologically reactive fragment thereof, or a peptide mimotope thereof.

6. The pathogen according to any one of claims 1-3 and 5, wherein the Helicobacter antigen is selected from the group consisting of the antigens AlpA, AlpB, immunologically reactive fragments thereof, or a peptide mimotope thereof.

7. The pathogen according to any one of claims 1-6,
wherein said nucleic acid molecule encoding a Helicobacter antigen is capable to be expressed phase variably.

8. The pathogen according to claim 7,
wherein said nucleic acid molecule encoding the Helicobacter antigen is under control of an expression signal which is substantially inactive in the pathogen and which is capable to be activated by a nucleic acid reorganization caused by a nucleic acid reorganization mechanism in the pathogen.

9. The pathogen according to claim 8,
wherein the expression signal is a bacteriophage promoter, and the activation is caused by a DNA reorganization resulting in the production of a corresponding bacteriophage RNA polymerase in the pathogen.

10. The pathogen according to any one of claims 1-9, further comprising at least one second nucleic acid molecule encoding an immunomodulatory polypeptide, wherein said pathogen is capable to express said second nucleic acid molecule.

11. Pharmaceutical composition comprising as an active agent a recombinant attenuated pathogen according to any one of claims 1-10, optionally together with pharmaceutically acceptable diluents, carriers and adjuvants.

12. Composition according to claim 11, which is a living vaccine, which is suitable for administration to a mucosal surface or via the parenteral route.

13. A method for the preparation of a living vaccine comprising formulating an attenuated pathogen according to any one of claims 1-10 in a pharmaceutically effective amount with pharmaceutically acceptable diluents, carriers and/or adjuvants.

14. A method for preparing a recombinant attenuated pathogen according to any one of claims 1-10, comprising the steps:
a) inserting a nucleic acid molecule encoding a Helicobacter antigen into an attenuated pathogen, wherein a recombinant attenuated pathogen is obtained, which is capable of expressing said nucleic acid molecule or is capable to cause expression of said nucleic acid molecule in a target cell, and
b) cultivating said recombinant attenuated pathogen under suitable conditions.

15. The method according to claim 14, wherein said nucleic acid molecule encoding a Helicobacter antigen is located on an extrachromosomal plasmid or inserted in the chromosome.

16. A method for identifying Helicobacter antigens, which raise a protective immune response in a mammalian host, comprising the steps of:
a) providing an expression gene bank of Helicobacter in an attenuated pathogen and
b) screening the clones of the gene bank for their ability to confer protective immunity against a Helicobacter infection in a mammalian host.
